SEQUENCE LISTING (19 sequences)

SEQ ID NO: 1 - DNA, 1498 bp, Homo sapiens
gcaaaggccaaggccagccaggacaccccctgggatcacactgagcttgccacatccccaaggcggccgaaccctccgcaaccaccagcccaggttaatccccagaggctccatggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccagtctttcaggtgtacccattgctcaactgtatggaggggatcccagggggctcaccatatgccggctgggcctacggcaagacggggctctaccctgcctcaactgtgtgtcccacccgcgaggactctcctccccaggccgtggaagatctggatggaaaaggcagcaccagcttcctggagactttgaagacagagcggctgagcccagacctcctgaccctgggacctgcactgccttcatcactccctgtccccaatagtgcttatgggggccctgacttttccagtaccttcttttctcccaccgggagccccctcaattcagcagcctattcctctcccaagcttcgtggaactctccccctgcctccctgtgaggccagggagtgtgtgaactgcggagcaacagccactccactgtggcggagggacaggacaggccactacctatgcaacgcctgcggcctctatcacaagatgaatgggcagaacaggcccctcatccggcccaagaagcgcctgattgtcagtaaacgggcaggtactcagtgcaccaactgccagacgaccaccacgacactgtggcggagaaatgccagtggggatcccgtgtgcaatgcctgcggcctctactacaagctacaccaggtgaaccggccactgaccatgcggaaggatggtattcagactcgaaaccgcaaggcatctggaaaagggaaaaagaaacggggctccagtctgggaggcacaggagcagccgaaggaccagctggtggctttatggtggtggctgggggcagcggtagcgggaattgtggggaggtggcttcaggcctgacactgggccccccaggtactgcccatctctaccaaggcctgggccctgtggtgctgtcagggcctgttagccacctcatgcctttccctggacccctactgggctcacccacgggctccttccccacaggccccatgccccccaccaccagcactactgtggtggctccgctcagctcatgagggcacagagcatggcctccagaggaggggtggtgtccttctcctcttgtagccagaattctggacaacccaagtctctgggccccaggcaccccctggcttgaaccttcaaagcttttgtaaaataaaaccaccaaagtcctg

SEQ ID NO: 2 - DNA, 1386 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccagtctttcaggtgtacccattgctcaactgtatggaggggatcccagggggctcaccatatgccggctgggcctacggcaagacggggctctaccctgcctcaactgtgtgtcccacccgcgaggactctcctccccaggccgtggaagatctggatggaaaaggcagcaccagcttcctggagactttgaagacagagcggctgagcccagacctcctgaccctgggacctgcactgccttcatcactccctgtccccaatagtgcttatgggggccctgacttttccagtaccttcttttctcccaccgggagccccctcaattcagcagcctattcctctcccaagcttcgtggaactctccccctgcctccctgtgaggccagggagtgtgtgaactgcggagcaacagccactccactgtggcggagggacaggacaggccactacctatgcaacgcctgcggcctctatcacaagatgaatgggcagaacaggcccctcatccggcccaagaagcgcctgattgtcagtaaacgggcaggtactcagtgcaccaactgccagacgaccaccacgacactgtggcggagaaatgccagtggggatcccgtgtgcaatgcctgcggcctctactacaagctacaccaggtgaaccggccactgaccatgcggaaggatggtattcagactcgaaaccgcaaggcatctggaaaagggaaaaagaaacggggctccagtctgggaggcacaggagcagccgaaggaccagctggtggctttatggtggtggctgggggcagcggtagcgggaattgtggggaggtggcttcaggcctgacactgggccccccaggtactgcccatctctaccaaggcctgggccctgtggtgctgtcagggcctgttagccacctcatgcctttccctggacccctactgggctcacccacgggctccttccccacaggccccatgccccccaccaccagcactactgtggtggctccgctcagctcatgagggcacagagcatggcctccagaggaggggtggtgtccttctcctcttgtagccagaattctggacaacccaagtctctgggccccaggcaccccctggcttgaaccttcaaagcttttgtaaaataaaaccaccaaagtcctg

SEQ ID NO: 3 - protein, 413 aa, Homo sapiens
MEFPGLGSLGTSEPLPQFVDPALVSSTPESGVFFPSGPEGLDAAASSTAPSTATAAAAALAYYRDAEAYRHSPVFQVYPLLNCMEGIPGGSPYAGWAYGKTGLYPASTVCPTREDSPPQAVEDLDGKGSTSFLETLKTERLSPDLLTLGPALPSSLPVPNSAYGGPDFSSTFFSPTGSPLNSAAYSSPKLRGTLPLPPCEARECVNCGATATPLWRRDRTGHYLCNACGLYHKMNGQNRPLIRPKKRLIVSKRAGTQCTNCQTTTTTLWRRNASGDPVCNACGLYYKLHQVNRPLTMRKDGIQTRNRKASGKGKKKRGSSLGGTGAAEGPAGGFMVVAGGSGSGNCGEVASGLTLGPPGTAHLYQGLGPVVLSGPVSHLMPFPGPLLGSPTGSFPTGPMPPTTSTTVVAPLSS

SEQ ID NO: 4 - DNA, 220 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccag

SEQ ID NO: 5 - DNA, 224 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactatactcagggacgctgaggcctacagacactccccag

SEQ ID NO: 6 - DNA, 235 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactaggcactggcctactacagggacgctgaggcctacagacactccccag

SEQ ID NO: 7 - DNA, 189 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgacactccccagtctttcaggtgtacccattgctcaactg

SEQ ID NO: 8 - DNA, 221 bp, Homo sapiens
atggagttccctggcctggggtccctggggacctcagagcccctcccccagtttgtggatcctgctctggtgtcctccacaccatgaatcaggggttttcttcccctctgggcctgagggcttggatgcagcagcttcctccactgccccgagcacagccaccgctgcagctgcggcactggcctactacagggacgctgaggcctacagacactccccag

SEQ ID NO: 9 - DNA, 218 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctagctgcggcactggcctactacagggacgctgaggcctacagacactccccag

SEQ ID NO: 10 - DNA, 204 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactatactcagggacgctga

SEQ ID NO: 11 - protein, 67 aa, Homo sapiens
MEFPGLGSLGTSEPLPQFVDPALVSSTPESGVFFPSGPEGLDAAASSTAPSTATAAAAALAYYTQGR

SEQ ID NO: 12 - DNA, 189 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactag

SEQ ID NO: 13 - protein, 62 aa, Homo sapiens
MEFPGLGSLGTSEPLPQFVDPALVSSTPESGVFFPSGPEGLDAAASSTAPSTATAAAAALAY

SEQ ID NO: 14 - DNA, 189 bp, Homo sapiens; identical to SEQ ID NO: 7

SEQ ID NO: 15 - protein, 64 aa, Homo sapiens
MEFPGLGSLGTSEPLPQFVDPALVSSTPESGVFFPSGPEGLDAAASSTAPSTLPSLSGVPIAQL

SEQ ID NO: 16 - DNA, 87 bp, Homo sapiens
atggagttccctggcctggggtccctggggacctcagagcccctcccccagtttgtggatcctgctctggtgtcctccacaccatga

SEQ ID NO: 17 - protein, 28 aa, Homo sapiens
MEFPGLGSLGTSEPLPQFVDPALVSSTP

SEQ ID NO: 18 - DNA, 198 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctagctgcggcactggcctactacagggacgctga

SEQ ID NO: 19 - protein, 65 aa, Homo sapiens
MEFPGLGSLGTSEPLPQFVDPALVSSTPESGVFFPSGPEGLDAAASSTAPSTATASCGTGLLQGR

(19) United States
(12) Patent Application Publication
(10) Pub. No.: US 2006/0003335 A1
(43) Pub. Date: Jan. 5, 2006

(54) Methods for diagnosing acute megakaryoblastic leukemia

(76) Inventor: John D. Crispino, Wilmette, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/880,764

(22) Filed: Jun. 30, 2004

(51) Int. Cl. C12Q 1/68

(52) U.S. Cl. 435/6

(57) ABSTRACT

The present invention is directed to methods and compositions for use in the diagnosis of acute megakaryoblastic leukemia. More particularly, it is shown that mutations in exon 2 of GATA-1 correlated with a predisposition to acute megakaryoblastic leukemia. Methods and compositions for exploiting this finding are described.

### FIELD OF THE INVENTION

The present invention is directed to methods and compositions for use in the diagnosis of acute megakaryoblastic leukemia.

### BACKGROUND

Trisomy 21, commonly known as Down syndrome (DS), is characterized by an extra copy of chromosome 21. Individuals afflicted with Down syndrome have severe mental retardation, reduced life expectancies, and abnormal immune responses that predispose them to serious infections as well as thyroid autoimmunity. Children with Down syndrome have a 10-20 fold increased risk of developing leukemia, in particular acute megakaryoblastic leukemia (AMKL; Lange, Br. J. Haematol., 110, 512-524, 2000). It is estimated that approximately 1 in 150 DS children will develop this malignancy by the age of three. DS children are also predisposed to another myeloid disease, termed transient myeloproliferative disorder (TMD, for review see Gamis and Hilden, J Pediatr Hematol Oncol, 24(1), 2-5, 2002). As many as 10% of DS infants develop TMD, in which immature megakaryoblasts accumulate in the bone marrow and peripheral blood. This disorder undergoes spontaneous remission in the majority of cases. Of note, approximately 30% of DS infants with TMD develop AMKL later in life. TMD blasts are morphologically indistinguishable from AMKL blasts, contributing to the hypothesis that the second disease is derived from the first (Lange, Br. J. Haematol., 110, 512-524, 2000; Taub and Ravindranath, J Pediatr Hematol Oncol, 24(1), 6-8, 2002). Often the karyotype of the AMKL blasts is more complex than that of TMD.

TMD spontaneously resolves in most cases, without therapeutic intervention. However, severe and sometimes fatal forms of TMD do occur, with hepatic fibrosis and liver dysfunction. Based on the liver infiltration and the spontaneous remission, it has been speculated that TMD may arise from fetal liver hematopoietic progenitors (Gamis and Hilden, J Pediatr Hematol Oncol, 24(1), 2-5, 2002). Treatment of severe forms of TMD involves administration of low doses of Ara-C, but the effective dose, the appropriate timing and frequency of drug delivery is still being evaluated. Unlike TMD, megakaryoblastic leukemia in DS is aggressively treated with chemotherapy, including infusion of low doses of Ara-C; there is an excellent prognosis for this malignancy, with nearly a 90% event free survival (Lange, Br. J. Haematol., 110, 512-524, 2000).

Despite the advances in treatment of these myeloid disorders in Down syndrome, little progress has been made in identifying the specific genetic factors that are involved. It is certainly likely that overexpression of a gene or genes on chromosome 21 is involved, with RUNX1/AML1 being a prime candidate. However, these factors remain undefined. In addition, it is likely that other genetic abnormalities contribute to the progression of the malignancies. Identification of these factors and the correlation with myeloid disorders will prove useful not only in the treatment of DS-AMKL but also may be useful in developing effective models and regimens for treating other types of blood disorders.

### SUMMARY OF THE INVENTION

The present invention is directed to methods of diagnosing transient myeloproliferative disorder (TMD) comprising obtaining a sample from a subject suspect of having a predisposition to TMD; and determining the loss or mutation of a GATA-1 gene in cells of the sample, wherein the loss or mutation of a GATA-1 gene, is diagnostic of TMD. The sample may be selected from the group consisting of blood, an amniocentesis sample, somatically in utero fetal blood, and bone marrow. More particularly, the sample is a tissue or fluid sample. The determining in this diagnostic method generally comprises assaying for a GATA-1 nucleic acid from the sample. In preferred embodiments, the method may further comprise subjecting the sample to conditions suitable to amplify the nucleic acid. In other embodiment, the method comprises the step of comparing the expression of GATA-1 in the sample with the expression of GATA-1 in non-TMD samples. More particularly, the comparison involves evaluating the level of GATA-1 expression, or evaluating the structure of the GATA-1 gene, protein or transcript. The evaluating method may use any technique commonly used for such evaluation, including but not limited to sequencing, wild-type oligonucleotide hybridization, mutant oligonucleotide hybridization, SSCP analysis, PCR, denaturing gradient gel electrophoresis and RNase protection. In particular embodiments, the evaluating comprises performing nucleic acid hybridization using an oligonucleotide derived from wild-type or mutant GATA-1 and the oligonucleotide is configured in an array on a chip or wafer. In particular embodiments, the diagnostic method is performed in an individual who has been diagnosed with a Down syndrome.

The diagnosing of TMD will likely involve use of a TMD sample which comprises a mutation in the coding sequence of GATA-1. More particularly, the mutation is one which produces a deletion mutant, an insertion mutant, a frameshift mutant, a nonsense mutant, a missense mutant or splice mutant of GATA-1. In preferred embodiments, there are disclosed herein frameshift mutation of the GATA-1 encoding DNA, wherein the mutation results in a premature termination of the GATA-1 gene product. Specifically contemplated are diagnostic methods and compositions that detect mutations exon 2 of GATA-1. These frameshift mutations may result from a from a deletion in codons 1 through to 83.

An exemplary mutation is one in which the frameshifts results in a STOP at codon 62 of wild-type GATA-1. In preferred aspects, the mutation produces mutant GATA-1 protein that is a shortened GATA-1 protein which lacks all or a portion of the N-terminal activation domain of wild-type GATA-1 protein. Preferably, the resultant mutant GATA-1 protein interacts with friend of GATA-1 to the same extent as full-length GATA-1, but has a reduced transactivation potential.

Another exemplary mutation is an insertion mutation which disrupts the open reading frame of GATA-1 after codon 62. Preferably, this disruption after codon 62 results in the introduction of a stop codon 6 residues after tyrosine 62. Another particular mutation is one in which there is a an insertion of TACT at 187-188 of GATA-1. In still another mutation, there is an insertion mutation is a 15 base pair insertion at 173-174 of GATA-1. Yet another exemplary mutation for use in the diagnostic methods involves an insertion mutation is an insertion of T at 84-85 of GATA-1, which results in a truncation of the GATA-1 protein product at Thr27. Yet another mutation comprises a deletion mutation in the open reading frame of GATA-1. More particularly, the deletion mutation is a deletion of 152-210 of the open reading frame of GATA-1, which truncates the GATA-1 protein product at Pro50. Another exemplary deletion mutation is a deletion of 166-167 of the open reading frame of GATA-1, which truncates the GATA-1 protein product at Ala55.

The present invention also may be used for diagnosing acute megakaryoblastic leukemia (AMKL) comprising obtaining a sample from a subject suspect of having AMKL; and determining the loss or mutation of a GATA-1 gene in cells of the sample, wherein the loss or mutation of a GATA-1 gene, is diagnostic of AMKL.

In other preferred aspects, the invention involves a method of distinguishing acute megakaryoblastic leukemia (AMKL) from other types of leukemia by obtaining a sample from a leukemia patient; and determining the loss or mutation of a GATA-1 gene in cells of the sample, wherein the loss or mutation of a GATA-1 gene, indicates the patient as having AMKL.

Also contemplated herein is an expression construct comprising a nucleic acid that encodes a mutant GATA-1 protein operably linked to a heterologous promoter, wherein the mutant GATA-1 protein is a GATA-1 protein which lacks all or a portion of the N-terminal activation domain of wild-type GATA-1 protein, but interacts with friend of GATA-1 to the same extent as full-length GATA-1. The expression construct may be one in which the heterologous promoter is selected from the group consisting of CMV, RSV, SV40, EF1α, pUB, TEF1, and tetracycline inducible promoter. The expression construct may comprise nucleic acids of a viral vector selected from the group consisting of retrovirus, adenovirus, adeno-associated virus, herpes virus, and vaccinia virus. In preferred embodiments, the expression construct comprises nucleic acid comprises a mutation selected from the group consisting of an insertion of TACT at 187-188 of wild-type GATA-1, a 15 nucleotides insertion at 173-174 of wild-type GATA-1, an insertion of T at 84-85 of wild-type GATA-1, a deletion of 152-210 of the open reading frame of wild-type GATA-1, and a deletion of 168-167 of the open reading frame of wild-type GATA-1.

Also contemplated herein are host cells expressing the nucleic acid of the expression construct of described above.

The invention further contemplates a method of making a mutant GATA-1 protein which lacks all or a portion of the N-terminal activation domain of wild-type GATA-1 protein, but interacts with friend of GATA-1 to the same extent as full-length GATA-1, comprising growing the recombinant host cell that expresses a nucleic acid of an expression construct of the invention in culture under conditions to allow production of the protein. The method may further comprise isolating the mutant GATA-1 protein from the recombinant host cell in culture.

Preferred embodiments of the present invention describes a mutant mouse generated from the breeding of a first parent mouse having a partial trisomy for the homologous region of human 21 q to a second parent mouse carrying a GATA-1 mutation, wherein the mutant mouse has a Down syndrome phenotype and carries a mutation in GATA-1. In specific embodiments, the first parent mouse is a Ts65Dn mouse. Alternatively, the first parent mouse is a Ts1Cje mouse. In specific embodiments, the second parent mouse is DneodHS mouse. The resultant mutant mouse has a leukemia.

Also provided are transgenic non-human animals, wherein the cells of the animal comprises a nucleic acid which encodes a mutant GATA-1 gene, under the control of a promoter, wherein the mutant GATA-1 gene produces a truncated GATA-1 protein that lacks the N-terminal activation domain of wild-type GATA-1. The transgenic non-human animal serves a model for leukemia. The transgenic animal is one in which the mutant GATA-1 gene comprises a mutation in exon 2. More particularly, the mutation is selected from the group consisting of an insertion of TACT at 187-188 of wild-type GATA-1, a 15 nucleotides insertion at 173-174 wild-type GATA-1, an insertion of T at 84-85 of wild-type GATA-1, a deletion of 152-210 of the open reading frame of wild-type GATA-1, and a deletion of 168-167 of the open reading frame of wild-type GATA-1.

Also provided herein is a microarray for measuring gene expression of GATA-1 comprising at least 10 oligonucleotides having distinct sequences derived from wild-type and mutant GATA-1. More specifically, the array comprises at least 15 oligonucleotide sequences. Preferably, the oligonucleotide sequences are derived from exon 2 of GATA-1. In preferred embodiments, at least one sequence of the microarray comprises the entire wild-type exon 2 and at least one other sequence of the microarray comprises a mutant exon 2 comprising a mutation selected from the group consisting of an insertion of TACT at 187-188 of GATA-1, a 15 nucleotides insertion at 173-174 of GATA-1, an insertion of T at 84-85 of GATA-1, a deletion of 152-210 of the open reading frame of GATA-1, and a deletion of 168-167 of the open reading frame of GATA-1.

The foregoing paragraphs are not intended to define every aspect of the invention, and additional aspects are described in other sections, such as the Detailed Description.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Although the applicants invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicants reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows the roles of GATA-1 and its cofactor FOG-1 in erythroid and megakaryocyte development.

FIG. 2 provides an analysis of GATA-1 mutations in Down syndrome related acute megakaryoblastic leukemia (DS-AMKL). FIG. 2a shows results from single strand conformation polymorphism assays. These assays were used to screen for mutations in GATA-1 in patients with either acute erythroleukemia (AML-M6), acute megakaryoblastic leukemia (AMKL), other de novo AML (P120), or non-megakaryoblastic AML evolving from myelodysplastic syndrome (P121, P128, P131). The arrows point to aberrantly migrating PCR products. FIG. 2b shows that direct sequencing of the DS-AMKL-1 aberrant PCR product resulted in double peaks after codon 62, indicating the presence of a frameshift mutation. The PCR products were then subcloned, and individual clones were re-sequenced. The cloned mutant allele had a 4 base pair insertion that altered the GATA-1 reading frame and resulted in the introduction of a stop codon six residues downstream of Tyr62. FIG. 2c shows the domain structure of GATA-1 depicting the N-terminal activation domain (AD) and the two highly conserved zinc fingers (Nf, Cf). Asterisks indicate the positions of the mutations in the six DS-AMKL patients, with the numbers corresponding to the DS-AMKL patients.

FIG. 3 shows RUNX1 (AML1) SSCP assays. The Runt domain of RUNX1 is not mutated in this cohort of DS-AMKL patients. SSCP analysis of five of the DS-AMKL samples and DNA from the CMK cell line (established from the malignant cells of a male DS-AMKL patient (Komatsu, N., Suda, T., Moroi, M., Tokuyama, N., Sakata, Y., Okada, M., Nishida, T., Hirai, Y., Sato, T., Fuse, A. and et al. (1989) Blood, 74(1), 42-8; Sato, T., Fuse, A., Eguchi, M., Hayashi, Y., Ryo, R., Adachi, M., Kishimoto, Y., Teramura, M., Mizoguchi, H., Shima, Y. and et al. (1989) Br J Haematol, 72(2), 184-90) indicated the presence of only one alteration in RUNX1 within exon 3 of the gene in patient DS-AMKL-1 (indicated by arrows). Sequencing revealed the presence of a single nucleotide change within exon 3 that is present in the overlapping PCR reactions 3.1 and 3.2. This change is in the wobble position of Val101, and does not result in an altered protein. The control DNAs were taken from healthy individuals.

FIG. 4a shows western blots of COS cells harvested after transient transfection with either wild-type (WT), the DS-AMKL representative mutant Tyr63Stop, or the FOG non-interacting Val205Gly mutant GATA-1. Nuclear extracts were probed with either the N6 or C-20 anti-GATA-1 antibodies, which recognize the N-terminus and C-terminus respectively. Both antibodies recognize the 50 kD full-length protein, while the shorter 40 kD version is recognized by only the C-20 antibody. FIG. 4b shows diagrammatic representation of the proteins encoded by GATA-1. Both the 50 kD and 40 kD forms are generated by the wild-type allele, while only the 40 kD protein is translated by the Tyr63Stop mutant allele. FIG. 4c shows western blot detected with the C-20 anti-GATA-1 antibody demonstrates that the human K562 cell line expresses both forms of GATA-1 (in both nuclear extract and cell lysate), while the blast cells in patient DS-AMKL-1 express only the 40 kD protein (lysates harvested from 1 and 2 million cells shown). The percentage of blasts in this sample was greater than 90%. FIG. 4d shows western blot stained with the C-20 anti-GATA-1 antibody revealed that, in contrast to K562, HEL and L8057 hematopoietic cells, CMK cells fail to express full length GATA-1. The CMK megakaryoblastic cell line, established from the malignant cells of a child with DS-AMKL, harbors a frame-shift mutation within exon 2 of GATA-1. Nuclear extracts from five million cells were used in this experiment. K562, HEL and CMK nuclear extracts were loaded in duplicate.

FIG. 5 shows Functional analysis of the 40 kD GATA-1s protein. FIG. 5a shows electrophoretic mobility shift assays demonstrate that the 40 kD GATA-1s protein translated from the Tyr63Stop allele binds the palindromic GATA-1 site to a similar extent as wild-type GATA-1. As expected, both the N6 and C-20 anti-GATA-1 antibodies supershifted the GATA-1-DNA complex, but only C-20 associated with the 40 kD GATA-1 s-DNA complex. FIG. 5b shows co-immunoprecipitation assays reveal that the shortened form of GATA-1 associated with FOG-1 in extracts from transfected COS cells to a similar degree as full length GATA-1. Nuclear extracts from transfected COS cells were immunoprecipitated with anti-Flag antibody and the resulting proteins, and original nuclear extracts, were visualized with either the C20 anti-GATA-1 or anti-Flag antibodies. The Val205Gly GATA-1 mutant, which has a reduced affinity for FOG-1, and the Flag-tagged FOG protein were previously described (Crispino, J. D., Lodish, M. B., MacKay, J. P. and Orkin, S. H. (1999) Mol Cell, 3(2), 219-28). FIG. 5c shows NIH3T3 cells were transfected with a reporter construct harboring 650 bp of the chicken GATA-1 promoter, which includes a GATA-1 response element, linked to the luciferase gene and with either wild-type, Tyr63Stop GATA-1, or the empty pXM vector. The transfections also included an equal amount of a β-galactosidase control DNA. The fold induction for each construct represent four independent experiments performed in duplicate, after normalization to β-galactosidase levels.

FIG. 6 shows a schematic of breeding protocol to introduce trisomy 16 into GATA-1 knock-down (mutant) mice. In round 1, hemizygous male GATA-1 knock down mice (XmY) are crossed with female Ts65Dn mice (XX+16). In the F1 generation, there are four possible genotypes, including heterozygous GATA-1 mutant females (XmX), with and without trisomy 16, as well as wild-type males (XY), with or without trisomy 16. In round 2, the F1 heterozygous GATA-1 mutants, with trisomy 16 (XmX, +16, bold) are crossed again to hemizygous GATA-1 mutants (XmY). The F2 generation will be comprised of 8 different genotypes, including homozygous mutant females (XmXm), heterozygous females, hemizygous males and wild-type males, each with and without trisomy 16. In round 3, the F2 homozygous GATA-1 mutant females are crossed with trisomy 16 (XmXm, +16, bold), to hemizygous male mutants once more. This final cross will bring the F3 generation into homozygosity for the GATA-1 mutation, with half the offspring having trisomy 21. This breeding scheme is necessarily complex, because male Ts65Dn mice are sterile. Female Ts65Dn mice are, however, fertile.

FIG. 7 shows targeting strategy to create a conditional mutant allele of GATA-1. The targeting construct will harbor a floxed pGK neomycin cassette within the intron preceding exon 2 (at the Avr II site), a single loxP site within the intron downstream of exon 2 (at the Bsa I site), and an HSV-tk cassette outside the flanking GATA-1 genomic sequences. The three loxP sites will be in the same orientation to facilitate correct excision. The targeting construct will be electroporated into male CJ7 ES cells, and G418 and gancyclovir resistant clones will be selected. Proper homologous recombinants can be screened for by Southern blot analysis, using the exon 5 sequences as a probe (solid line above exon 5, top). Since the Bsa I site is destroyed by the loxP insertion, correctly targeted clones will have a 12 Kb fragment following Xba I, Bsa I double digestion, while the wild-type clone will harbor a 7.5 Kb fragment. Once the correctly targeted clones are identified, the neomycin selectable marker is easily removed by transient expression of Cre recombinase. Clones that have recombined the two loxP sites flanking the pGK-neomycin cassette can be identified by screening for loss of the G418 resistance and Southern blot analysis. A 10 Kb fragment should be detectable following the Xba I, Bsa I double digestion. The final clones that harbor two lox P sites flanking exon 2 can then be injected into C57B1/6 blastocysts to generate chimeras. X, Xba I, Bs, Bsa I; Bg, Bgl II; A, Avr II.

### DETAILED DESCRIPTION OF THE PREFERRED EMBODIMENTS

Mutations in transcription factors, such as RUNX1 and CEBPA, have been reported to be involved in human malignancies (for a brief review, see ref. Look, A. T. (2002) Nat Genet, 32(1), 83-4). A transcription factor that plays an important role in normal hematopoiesis is GATA-1. This transcription factor also is known to contain numerous missense mutations, many of which have been shown to correlate to certain blood disorders. Inherited mutations in the N-terminal zinc finger domain of GATA-1, which disrupt the interaction between GATA-1 and FOG-1, cause several types of congenital dyserythropoietic anemia and thrombocytopenia (Nichols, K. E., Crispino, J. D., Poncz, M., White, J. G., Orkin, S. H., Maris, J. M. and Weiss, M. J. (2000) Nat Genet, 24(3), 266-70; Freson, K., Devriendt, K., Matthijs, G., Van Hoof, A., De Vos, R., Thys, C., Minner, K., Hoylaerts, M. F., Vermylen, J. and Van Geet, C. (2001) Blood, 98(1), 85-92; Mehaffey, M. G., Newton, A. L., Gandhi, M. J., Crossley, M. and Drachman, J. G. (2001) Blood, 98(9), 2681-8). Also, mutations in GATA-1 that interfere with normal DNA binding by the N-finger of GATA-1 have been associated with dyserythropoiesis in humans (Yu, C., Niakan, K. K., Matsushita, M., Stamatoyannopoulos, G., Orkin, S. H. and Raskind, W. H. (2002) Blood, 100(6), 2040-5).

In the present application, the inventors have shown that mutations in GATA-1 result in other hematopoietic diseases and further, that somatic mutations in GATA-1 contribute to myelodysplastic syndromes or acute myeloid leukemias. More specifically, the inventors discovered that mutations in GATA-1 participate in one form of AML, the megakaryoblastic leukemia associated with Down syndrome. The inventors found that GATA-1 was mutated in all of the DS-AMKL samples tested but did not find mutations in GATA-1 in leukemic cells of DS patients with other types of acute leukemia, or in other patients with AMKL who did not have DS. Furthermore, there were no GATA-1 mutations in DNA samples from other patients with acute leukemia or healthy individuals. In addition, it was determined that mutations are somatically acquired. These observations showed that disruption of normal GATA-1 function is an essential step in the initiation or progression of megakaryoblastic leukemia in DS. These findings provide the first correlation of the characterization of specific genetic alterations that are associated with this malignancy.

Discussed in further detail herein below are the mechanisms by which mutations in GATA-1 lead to abnormal blood cell development and pathogenesis of AMKL. In addition, the correlation of GATA-1 mutations to a “pre-leukemia” of Down syndrome, named Transient Myeloproliferative Disorder (TMD) (4) also is discussed. As many as 30% of infants with TMD will develop AMKL within three years, but there are currently no prognostic factors that predict the future onset of AMKL. With the findings of the present invention, GATA-1 mutations may be used to screen for those individual having TMD patients who will later develop AMKL, thereby identifying those children most at risk for future malignancies. Furthermore, the present findings provide insights into the role of GATA-1 in normal hematopoiesis, disorders of blood cell production, and leukemogenesis, and will likely serve as a useful model for the exploration of potential mechanisms of leukemic transformation. Also described are methods and compositions for making and using animal models of DS-AMKL. Such models may be used in assays to identify factors that cooperate with loss of wild-type GATA-1 in leukemogenesis and to develop agents for the therapeutic intervention of AMKL and TMD.

A. Involvement of GATA-1 in Normal and Aberrant Hematopoiesis

The present section provides a brief description of the involvement of GATA-1 in blood cell development to the extent that such a description will facilitate a better understanding of the methods and compositions of the present invention.

Human GATA-1 is also called Gf-1 and is available to those of skill in the art at Genbank Accession No. NM_{—}002049 (reproduced herein as SEQ ID NO: 1, encoding GATA-1 transcription factor of SEQ ID NO:3). The full length human GATA-1 cDNA sequence is 1498 nucleotides in length. For the purposes of the present application, the GATA-1 sequence was renumbered with the first nucleotide of the coding sequence being designated nucleotide residue 1, this renumbered GATA-1 nucleotide sequence is depicted as SEQ ID NO:2. In the Examples presented herein below, the inventors showed that there were various mutation in the GATA-1 nucleotide sequence in individuals with AMKL. For example, using the numbering of SEQ ID NO:3, the inventors discovered that the GATA-1 sequence of AMKL patients contained one or more mutations selected from the group consisting of an insertion of TACT at 187-188 of wild-type GATA-1 (SEQ ID NO:10), a 15 nucleotides insertion at 173-174 of wild-type GATA-1 (SEQ ID NO:6), an insertion of T at 84-85 of wild-type GATA-1 (SEQ ID NO:8), a deletion of 152-210 of the open reading frame of wild-type GATA-1 (SEQ ID NO:7), and a deletion of 167-168 of the open reading frame of wild-type GATA-1 (SEQ ID NO:9). These and other mutations in GATA-1 may be used in the diagnostic applications of the present invention. Other mutations that are contemplated to be particularly useful herein will be those mutations that result in the loss or decreased expression of exon 2 of GATA-1.

GATA-1 is expressed primarily in erythroid cells, megakaryocytes, eosinophils and mast cells, where a large number of genes harbor GATA DNA binding motifs. Studies in mice that completely lack GATA-1 expression (GATA-1 null mice) have shown that GATA-1 is essential for the proper development of erythroid cells (Fujiwara, Y., Browne, C. P., Cunniff, K., Goff, S. C. and Orkin, S. H. (1996) Proc Natl Acad Sci USA, 93(22), 12355-8). GATA-1 null erythroid cells are blocked in the maturation at the proerythroblast stage, and subsequently undergo apoptosis. As a consequence, GATA-1 null mice die in mid-gestation of anemia. Other knockout studies have shown that GATA-1 is required for the proper maturation of megakaryocytes (McDevitt, M. A., Shivdasani, R. A., Fujiwara, Y., Yang, H. and Orkin, S. H. (1997) Proc Natl Acad Sci USA, 94(13), 6781-5; Shivdasani, R. A., Fujiwara, Y., McDevitt, M. A. and Orkin, S. H. (1997) Embo J, 16(13), 3965-73; Takahashi, S., Komeno, T., Suwabe, N., Yoh, K., Nakajima, O., Nishimura, S., Kuroha, T., Nagasawa, T. and Yamamoto, M. (1998) Blood, 92(2), 434-42). In the absence of GATA-1, megakaryocytes proliferate excessively and fail to generate platelets (Shivdasani, R. A., Fujiwara, Y., McDevitt, M. A. and Orkin, S. H. (1997) Embo J, 16(13), 3965-73; Vyas, P., Ault, K., Jackson, C. W., Orkin, S. H. and Shivdasani, R. A. (1999) Blood, 93(9), 2867-75). Furthermore, abnormal megakaryocytes accumulate in the spleen and bone marrow of GATA-1 ‘knock-down’ mice, resulting in anemia and thrombocytopenia (Shivdasani, R. A., Fujiwara, Y., McDevitt, M. A. and Orkin, S. H. (1997) Embo J, 16(13), 3965-73). Loss of GATA-1 also has been shown to result in the development of myelodysplastic syndrome and premature death of the animals (Takahashi, S., Komeno, T., Suwabe, N., Yoh, K., Nakajima, O., Nishimura, S., Kuroha, T., Nagasawa, T. and Yamamoto, M. (1998) Blood, 92(2), 434-42). GATA-1 also is necessary for the development of eosinophils (Hirasawa, R., Shimizu, R., Takahashi, S., Osawa, M., Takayanagi, S., Kato, Y., Onodera, M., Minegishi, N., Yamamoto, M., Fukao, K., Taniguchi, H., Nakauchi, H. and Iwama, A. (2002) J Exp Med, 195(11), 1379-86; Yu, C., Cantor, A. B., Yang, H., Browne, C., Wells, R. A., Fujiwara, Y. and Orkin, S. H. (2002) J Exp Med, 195(11), 1387-95).

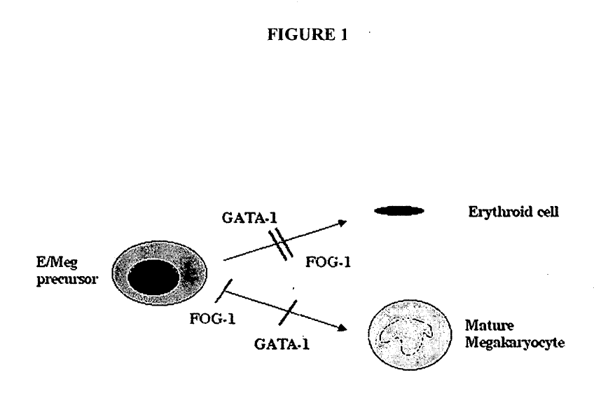

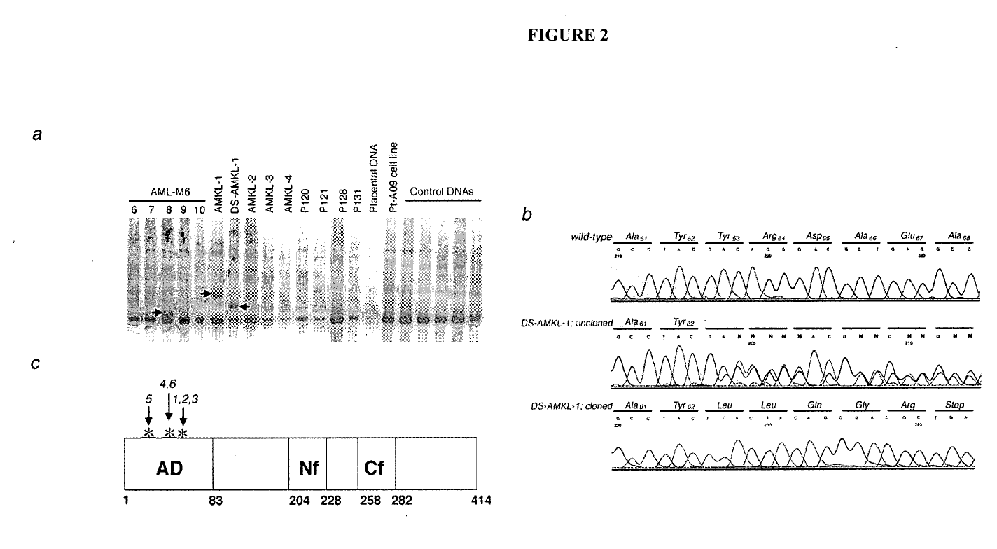

GATA-1 has three functional domains: an N-terminal transactivation domain and two zinc fingers (refer to FIG. 2c for a schematic representation). The C-terminal zinc finger is required for binding of GATA-1 to DNA, while the N-terminal zinc finger stabilizes binding to a subset of sites, termed palindromic motifs (Trainor, C. D., Omichinski, J. G., Vandergon, T. L., Gronenbom, A. M., Clore, G. M. and Felsenfeld, G. (1996) Mol Cell Biol, 16(5), 2238-47). In addition to binding DNA, the N-finger plays an important role by recruiting a cofactor named Friend of GATA-1 (FOG-1; ref. Tsang, A. P., Visvader, J. E., Turner, C. A., Fujiwara, Y., Yu; C., Weiss, M. J., Crossley, M. and Orkin, S. H. (1997) Cell, 90(1), 109-19). Mice that lack FOG-1 exhibit an erythroid defect similar to that of GATA-1 null mice, but have an earlier block in megakaryocyte development (Tsang, A. P., Fujiwara, Y., Hom, D. B. and Orkin, S. H. (1998) Genes Dev, 12(8), 1176-88). FIG. 1 summarizes the known stages in red cell and megakaryocyte maturation during which GATA-1 and FOG-1 are required. While both genes are critical for terminal differentiation of erythroid cells, FOG-1 null erythroid precursors survive longer than those lacking GATA-1, suggesting that GATA-1 may have a FOG-1-independent function (Tsang, A. P., Fujiwara, Y., Hom, D. B. and Orkin, S. H. (1998) Genes Dev, 12(8), 1176-88). Although it was initially believed that FOG-1 had a GATA independent function in early megakaryocyte specification, it has recently been shown that FOG-1 can function by interacting with either GATA-1 or GATA-2 in this role (Chang, A. N., Cantor, A. B., Fujiwara, Y., Lodish, M. B., Droho, S., Crispino, J. D. and Orkin, S. H. (2002) Proc Natl Acad Sci USA, 99(14), 9237-42). Taken together, these studies show that both GATA-1 and FOG-1 act as key regulators in the development of blood cells.

While it is known that both zinc fingers of GATA-1 are essential for normal activity, it is less clear what role the N-terminal activation domain plays in development. This region was initially defined in transient reporter assays in fibroblasts (Martin, D. I. and Orkin, S. H. (1990) Genes Dev, 4(11), 1886-98). However, the results of two studies have suggested that this domain is not required for GATA-1 function. First, GATA-1 molecules that lack the N-terminal activation domain can rescue the differentiation of GATA-1 deficient erythroid cell line (Weiss, M. J., Yu, C. and Orkin, S. H. (1997) Mol Cell Biol, 17(3), 1642-51). Second, the activation domain was dispensable in the conversion of an early myeloid cell line, 416B, to megakaryocytes (Visvader, J. E., Crossley, M., Hill, J., Orkin, S. H. and Adams, J. M. (1995) Mol Cell Biol, 15(2), 634-41). More recently, Masayuki Yamamoto's group has performed a detailed structure-function study of GATA-1 domains in vivo (Shimizu, R., Takahashi, S., Ohneda, K., Engel, J. D. and Yamamoto, M. (2001) Embo J, 20(18), 5250-60). In this line of experiments, researchers bred mice harboring wild-type and mutant GATA-1 transgenes to the GATA-1.05 mice. While the GATA-1.05 mice with no transgene died in mid-gestation of anemia, GATA-1.05 mice that harbor a wild-type GATA-1 transgene were born at the expected frequency and were healthy. As expected, GATA transgenes that lacked either zinc finger failed to rescue the embryonic lethality of GATA-1.05 mice. Surprisingly, however, mice that expressed a GATA-1 transgene without the N-terminal activation domain survived, but only when the transgene was expressed at very high levels. These findings suggest that the N-terminal activation domain does have a critical function in blood cell development.

B. Making and Using Transgenic Animals of the Invention

Particular aspects of the present invention involve the production of transgenic animals. In particular, the first set of transgenic mice contemplated by the present invention are those which have a loss of GATA-1 phenotype, in conjunction with trisomy 16 (mouse homologue of human chromosome 21). The second set of transgenic mice are those that conditionally express GATA-1s but not GATA-1. The rationale and methods and compositions for the production of these transgenic animals are provided in further detail herein below.

Those of skill in the art are aware of general techniques for making transgenic animals. Such techniques involve the integration of a given nucleic acid construct into the genome in a manner that permits the expression of a transgene or the knockout of an existing gene. Methods for producing transgenic animals are generally described by Wagner and Hoppe (U.S. Pat. No. 4,873,191; which is incorporated herein by reference), Palmiter and Brinster Cell, 41(2):343-5, 1985; which is incorporated herein by reference in its entirety) and in “Manipulating the Mouse Embryo; A Laboratory Manual” 2^{nd} edition (eds. Hogan, Beddington, Costantimi and Long, Cold Spring Harbor Laboratory Press, 1994; which is incorporated herein by reference in its entirety). In the present application, the genes of interest are GATA-1 related genes. The wild-type GATA-1 sequence is well known to those of skill in the art (see e.g., NM_{—}002049), as may be used as the underlying sequence for the production of the transgenic mice.

Typical techniques for producing transgenic animals involve the transfer of genomic sequences by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Methods for the production and purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); the Qiagenologist, Application Protocols, 3^{rd} edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

As discussed above, the inventors found that DNA samples from patients with DS-AMKL harbored frame-shift mutations within exon 2 of GATA-1 that blocked expression of full length GATA-1, but allowed for production of the shortened, N-terminal deleted protein GATA-1 s. GATA-1 mutations were not present in any non-DS AMKL subtypes. Thus, overexpression of a gene or genes in the critical region of 21 q may be an essential cooperating factor in the leukemic transformation. The first set of transgenic animals of the present invention provide a model for determining whether loss of GATA-1, in conjunction with trisomy 16 (mouse homologue of human chromosome 21), is sufficient for leukemic transformation.

GATA-1 knock down mice have been described in the art (McDevitt, M. A., Shivdasani, R. A., Fujiwara, Y., Yang, H. and Orkin, S. H. (1997) Proc Natl Acad Sci USA, 94(13), 6781-5; Shivdasani, R. A., Fujiwara, Y., McDevitt, M. A. and Orkin, S. H. (1997) Embo J, 16(13), 3965-73). These mice harbor a deletion in a hypersensitive site of the GATA-1 promoter and display reduced expression of GATA-1 in erythroid cells, and no detectable GATA-1 in the megakaryocytic lineage (McDevitt, M. A., Shivdasani, R. A., Fujiwara, Y., Yang, H. and Orkin, S. H. (1997) Proc Natl Acad Sci USA, 94(13), 6781-5; Shivdasani, R. A., Fujiwara, Y., McDevitt, M. A. and Orkin, S. H. (1997) Embo J, 16(13), 3965-73). Hemizygous males and homozygous females that survive to birth exhibit a normal hematocrit, but have a chronic thrombocytopenia (average platelet count of 10% of normal). An interesting phenotype of these GATA-1 knock-down mice was recently described. Vannucchi et al., recently described that at approximately 15 months of age, male mutant mice exhibit marked fibrosis of the bone marrow, with accumulation of immature megakaryocytes, extramedullary hematopoiesis in the liver, and severe anemia (Vannucchi, A. M., Bianchi, L., Cellai, C., Paoletti, F., Rana, R. A., Lorenzini, R., Migliaccio, G. and Migliaccio, A. R. (2002) Blood, 100(4), 1123-32). The mice eventually succumb to anemia and secondary disorders. Importantly, these mice do not develop acute myeloid leukemia of any kind. These GATA-1 knock-down mice do, however, provide an excellent model to study the effects of the complete loss of GATA-1.

In the present application, the GATA-1 knock-down mice are exploited in two different types of studies. In the first series of modifications, induction of megakaryoblastic leukemia is studied in the GATA-1 knock-down mice by breeding the GATA-1 knock down mice to animals that model Down syndrome (e.g., the Ts65Dn strain; available from Jackson labs). The progeny of these breeding experiments are compound mice that can be used to model and investigate the development of acute megakaryoblastic leukemia.

In a second line of modifications, bone marrow harvested from single and compound DS-GATA-1 knock-down mice is cultured in vitro and compared to assess the growth and morphology of megakaryocyte colonies. Wild-type and various GATA-1 mutant genes are introduced into these cells by retroviral infection, and megakaryocyte colony formation is determined in vitro. In preferred examples, overexpression of short GATA-1 isoform is achieved in order to determine whether such overexpression results in immortalization of megakaryocyte progenitors. In addition assays are performed to assess whether DS GATA-1 knock-down bone marrow progenitors, engineered to express mutant forms of GATA-1, will generate a leukemic phenotype in irradiated recipient animals. These studies provide one approach to characterize the growth of megakaryocyte progenitors that have GATA-1 deficiencies accompanying mouse trisomy 16, and may well serve as a model for DS-AMKL.

To introduce trisomy 21 into the GATA-1 knock down mice, and to address the mechanisms by which mutations in GATA-1 promote leukemogenesis in DS, the GATA-1 knock-down mice are bred with mice that model Down syndrome to generate compound mutants. The compound mutants preferably will be mutants that are predisposed to developing leukemia. In order to achieve these mutants, the preferred approach will be to utilize DS mice with a large chromosomal duplication rather than mice transgenic for a single gene on mouse chromosome 16 (which is homologous to human chromosome 21).

Presently, there are two mouse models of Down syndrome with segmental trisomy for the distal end of mouse chromosome 16 (see ref. Reeves, R. H., Baxter, L. L. and Richtsmeier, J. T. (2001) Trends Genet, 17(2), 83-8 for review). The first model, which has an extra copy of a region that spans 15.6 Mb and is estimated to contain 108 of the 225 genes from human chromosome 21 has been named Ts65Dn (Davisson, M. T., Schmidt, C., Reeves, R. H., Irving, N. G., Akeson, E. C., Harris, B. S. and Bronson, R. T. (1993) Prog Clin Biol Res, 384, 117-33). These mice survive to adulthood and exhibit many of the symptoms of Down syndrome, including neural cognitive deficits, and craniofacial abnormalities. In addition, the mice have spatial learning and memory defects as well as behavioral abnormalities. However, the mice have not been reported to exhibit other features commonly found in DS, including congenital heart defects and the predisposition to leukemia. A second segmental trisomy 16 model, named Ts1Cje, spans a smaller region of chromosome 16, containing a 9.8 Mb region with 79 genes, and has been reported to exhibit fewer DS characteristics as compared to Ts65Dn mice (Sago, H., Carlson, E. J., Smith, D. J., Kilbridge, J., Rubin, E. M., Mobley, W. C., Epstein, C. J. and Huang, T. T. (1998) Proc Natl Acad Sci US A, 95(11), 6256-61).

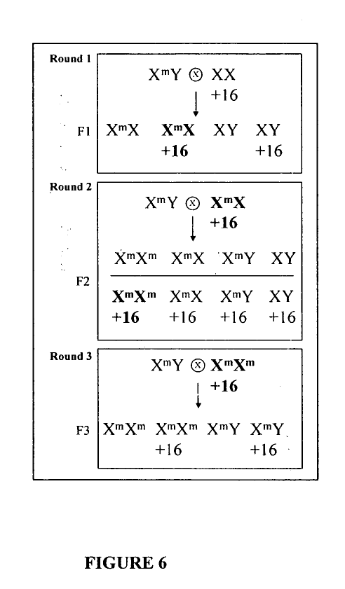

The Ts65Dn mice contain a greater number of genes that are likely involved in DS, and are commercially available (Jackson Laboratories). These mice are bred according to the breeding scheme outlined in FIG. 6. In humans and mice, GATA-1 is an X-linked gene. Briefly, in the first round of breeding, the male hemizygous GATA-1 knock-down mutants are crossed to female Ts65Dn mice. Offspring of four different genotypes are expected: 50% female heterozygous GATA-1 mutants, half with trisomy 16, and half without trisomy 16, plus 50% wild-type GATA-1 males, half with trisomy 16 and half without trisomy 16.

In the second generation, the male hemizygous GATA-1 knock-down mutants are crossed to the F2 female heterozygous GATA-1 mutants with trisomy 16. Of the eight different types of pups predicted, one group will be the most useful for the third round: female mice homozygous for the GATA-1 deficiency that also have trisomy 16. Mice of this genotype are bred with male hemizygous GATA-1 knock-down mutants. This cross will bring the GATA-L mutation into the homozygous state, which will allow for easier genotyping and for an accumulation of mutant mice. The karyotype classification is discussed in further detail below. In addition, the genotyping of the GATA-1 locus by Southern blot analysis from tail DNA is performed as previously described (Shivdasani, R. A., Fujiwara, Y., McDevitt, M. A. and Orkin, S. H. (1997) Embo J, 16(13), 3965-73).

The F3 generation of mice from FIG. 6 are: 1) Females homozygous for the GATA-1 deficiency that have trisomy 16; 2) Females homozygous for the GATA-1 deficiency without trisomy 16; 3) Males hemizygous for the GATA-1 deficiency with trisomy 16; and 4) males hemizygous for the GATA-1 deficiency without trisomy 16. Twenty mice of each genotype are monitored in order to determine whether the combination of GATA-1 deficiency and the presence of trisomy 21 result in megakaryoblastic leukemia.

The mice are followed for up to 12 months to assess whether leukemia develops. At weaning, the mice are tail clipped for genotyping purposes and also ear tagged for identification. In addition, 100 μl peripheral blood from the retroorbital sinus is harvested for both karyotype classification and for establishing the baseline hematologic values. Although the mice are expected to suffer from thrombocytopenia, the platelet counts are within the safe range for routine sampling. Karyotypic classification of the mice (disomy 16 vs. trisomy 16) will be accomplished by fluorescence in situ hybridization (FISH) of murine bacterial artificial chromosome (BAC) clones containing genes from the proximal and distal region of chromosome 16 syntenic to human 21q. Cytospin slides will be prepared from 10 μl of blood. FISH is performed using techniques known to those of skill in the art (Espinosa, R. and Le Beau, M. M. (1997) Methods Mol. Biol., 68, 53-76). Briefly, BAC RP23-36112 (containing the Runx1/Cbfa2 gene) is labeled by nick translation with Bio-11-dUTP and detected with fluorescein-conjugated avidin, and BAC RP23-451M23 (containing the Dscam gene) is labeled with digoxigenin-11-dUTP and detected by incubation with rhodamine-conjugated sheep anti-digoxigenin antibodies. Slides are examined with a fluorescein/rhodamine double-bandpass filter set, and the numbers of red and green signals are scored in 100 interphase cells. The chromosomes are counterstained with DAPI.

The mice are closely monitored for signs of development of leukemia in several ways. This will include weighing each mouse twice a week, and observing the mouse's mobility, ability to reach food and water, and grooming habits. In addition, the mice are phlebotomized every two months to monitor the CBC, and also to analyze stained blood smears for the presence of abnormal blasts in the peripheral blood. If blasts are detected, then the appearance of the cells is assessed by morphology in stained cytospins as well as by reactivity against von Willebrand Factor (vWF) and against the megakaryocyte surface antigen CD41. Following sacrifice of the mouse, organs such as the spleen, bone marrow, thymus and other internal organs are harvested and assessed for histology, and blood for flow cytometry. The Bethesda proposals for classification of non-lymphoid hematopoietic neoplasms in mice, which was recently published in Blood (Kogan et al., Blood, 100(1): 238-245, 2002) may be used to classify the neoplasia. Any mouse malignancies may be further characterized using tissue necropsy, histology and flow cytometry determinations.

Any mice generated from the above protocol that have trisomy 16 and a GATA-1 deficiency that develop leukemia will be used to identify the critical region of mouse chromosome 16, and by extension, human chromosome 21. One of the proposed mutant crosses will cross the GATA-1 knock-down mice to the second DS model Ts1Cje, which has trisomy for fewer genes than Ts65Dn mice. If leukemia also arises in these compound mutant mice, it follows that the gene is also present in the narrowed region of mouse chromosome 16. If the leukemia cannot be reproduced in this DS background, then the susceptibility gene likely lies in the region outside of the Ts1Cje segment, but within the Ts65Dn segment. To narrow the region further, additional smaller segments of chromosome 16 may be used.

Megakaryocytes that lack GATA-1 exhibit altered growth properties both in vivo and in vitro. Compared to their normal counterparts, GATA-1-deficient primary megakaryocytes exhibit significant hyperproliferation when grown in liquid culture, and morphologically are small with retarded nuclear and cytoplasmic development (Vyas, P., Ault, K., Jackson, C. W., Orkin, S. H. and Shivdasani, R. A. (1999) Blood, 93(9), 2867-75). In addition, GATA-1 knock-down mice exhibit a significant increase in megakaryocytes in the spleen and bone marrow, with the cells displaying gross abnormalities including a small cytoplasm and excess smooth endoplasmic reticulum (Shivdasani, R. A., Fujiwara, Y., McDevitt, M. A. and Orkin, S. H. (1997) Embo J, 16(13), 3965-73). Methylcellulose colony assays from yolk sac and fetal livers of GATA-1 knock-down mice revealed that megakaryocyte progenitor numbers are normal, but that some megakaryocyte progenitors exhibit marked hyperproliferation, producing abnormally large colonies composed of immature megakaryocytes. Despite this, the mice do not develop megakaryoblastic leukemia. Consistent with this observation, GATA-1-deficient progenitors have not been reported to exhibit an increased self-renewal in culture. Immortalization of hematopoietic progenitors is widely used as an indication of leukemic transformation, as such it can be used to assess whether the introduction of trisomy 16 into the GATA-1 knock-down megakaryocytes has any effect on their self-renewal capacity in vitro (and in vivo). Similar experiments have demonstrated the leukemic transforming activity of a variety of leukemic fusion proteins, including AML-1/ETO (Okuda, T., Cai, Z., Yang, S., Lenny, N., Lyu, C. J., van Deursen, J. M., Harada, H. and Downing, J. R. (1998) Blood, 91(9), 3134-43; Higuchi, M., O'Brien, D., Kumaravelu, P., Lenny, N., Yeoh, E. J. and Downing, J. R. (2002) Cancer Cell, 1(1), 63-74), MLL-AFX (So, C. W. and Cleary, M. L. (2002) Mol Cell Biol, 22(18), 6542-52), and MLL-ELL (Lavau, C., Luo, R. T., Du, C. and Thirman, M. J. (2000) Proc Natl Acad Sci USA, 97(20), 10984-9).

In order to perform the above assessments, the bone marrow from the following four groups of mice between 8 and 12 weeks of age is harvested:
1. male GATA-1 knock-down mice, with trisomy 16,
2. male GATA-1 knock-down littermates that lack trisomy 16,
3. male wild-type littermates with trisomy 16,
4. male wild-type littermates.

In all cases, bone marrow from male mice is harvested because males harbor only one allele of GATA-1. Forty-eight hours prior to harvest, the mice are treated with 5-FU (5-fluorouracil; 150, mg/kg) to enrich for primitive progenitors in the bone marrow. The donor mice are sacrificed and bone marrow cells isolated from both femurs by flushing the cells into media containing 5% serum. Mononuclear cells by Ficoll gradient and cultured in vitro in methylcellulose for 7 days. For such in vitro cultures, either methylcellulose media purchased from Stem Cell Technologies (Vancouver, Canada), or similar methylcellulose may be used. For megakaryocyte cultures, 100,000 unpurified bone marrow progenitors are plated per 35 mm dish of methylcellulose containing thrombopoietin (tpo, 50 ng/ml), IL-6 (20 ng/ml), IL-3 (10 ng/ml) and IL-11 (50 ng/ml), in duplicate. This mixture of four cytokines provides the optimal mix for megakaryocyte development, with approximately 40-50 CFU-Mks (Colony forming unit—megakaryocyte) generated per 100,000 cells plated. After 7 days, the number of megakaryocyte colonies are counted and several individual clones are used to prepare slides by cytospin preparation. One set of slides is stained with May Grunwald Giemsa and a second with reagents to detect expression of acetylcholinesterase (AChE), which is abundantly expressed by mouse, but not human, megakaryocytes. In the next step, the remaining colonies are harvested from methylcellulose media and used to prepare single cell suspensions by passage through a 21 G needle. 10,000 of these cells are then plated in methylcellulose media as above. The growth of CFU-Mks is assessed for another 7 days, and the number of colonies formed in this second generation determined. The colonies are again assessed for cellular morphology and expression of AChE. This serial replating is used for as many generations as the CFU-Mk colonies are produced, e.g., up to ten generations. Cells with either trisomy 16 alone, or the GATA-1 knock-down mutation alone will not exhibit a significant difference in the self-renewal capacity, but ones with both trisomy 16 and GATA-1 mutation may indeed exhibit an immortalized phenotype.

In yet another method, the present application introduces wild type, and various mutant forms of GATA-1 into GATA-1 knock-down bone marrow progenitors by retroviral transduction assay for transformation by GATA-1 mutants. These studies are performed to assay the ability of mutant forms of GATA-1 to induce immortalization. These studies are conducted as described above, except that following harvest from bone marrow, the progenitor cells are infected with retroviruses that express either wild-type or mutant forms of GATA-1. The progenitor cells for this study will be obtained from the following lines:
1. GATA-1 knock-down trisomy 16 mice,
2. GATA-1 wild-type trisomy 16 littermates,
3. GATA-1 knock-down, without trisomy 16,
4. wild-type littermates, without trisomy 16.

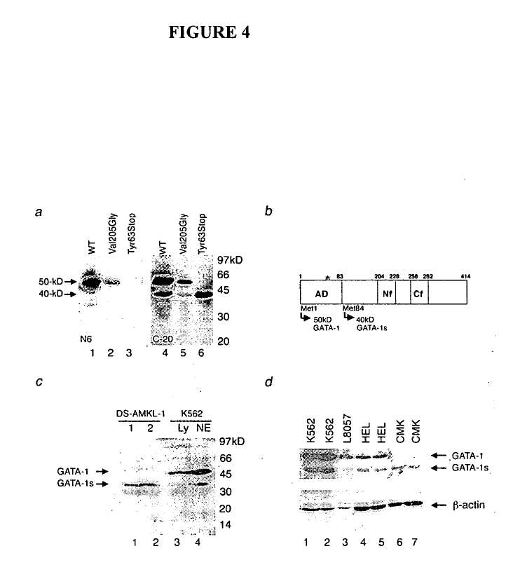

Constructs that harbor full-length wild type GATA-1 and a representative DS-AMKL mutant gene found in 3/6 patients, named Tyr63Stop, in the MSCV-neo vector will be used for this retroviral transfection. The Tyr63 Stop allele, which has a single point mutation that converts the tyrosine residue at amino acid 63 to a stop codon, was used in mammalian transfection assays and shown to encode for the GATA-1s isoform exclusively (See Examples and FIG. 4). Additional GATA-1 mutants may be created to mirror other mutations found in DS-AMKL patients. Bone marrow progenitors from GATA-1 knock-down mice that have trisomy 16 and have been engineered to express GATA-1s will exhibit an immortalized phenotype in vitro.

In order to perform the retroviral transfection, Bosc23 producer cells (Pear, W. S., Nolan, G. P., Scott, M. L. and Baltimore, D. (1993) Proc Natl Acad Sci USA, 90(18), 8392-6) are transfected with the MSCV constructs to generate helper free ecotropic retroviral supernatants. The isolated bone marrow cells are infected by the “spinoculation” procedure (Bahnson, A. B., Dunigan, J. T., Baysal, B. E., Mohney, T., Atchison, R. W., Nimgaonkar, M. T., Ball, E. D. and Barranger, J. A. (1995) Journal Virological Methods, 54, 131-143): a standard method that involves centrifugation of the recipient cells with retroviral particles. To monitor for expression of either wild-type GATA-1 or GATA-1 s, immunofluorescence may be performed on a small proportion of transduced bone marrow cells. The infected bone marrow progenitors are cultured in the megakaryocyte methylcellulose mix discussed above, except that the mix also includes G418 to select for progenitors that have been transduced by the retrovirus. After 7 days of growth, the cells are assayed for CFU-Mk formation by enumeration of colonies and by harvesting individual clones for cytospin preparation and staining. The primary colonies are then serially replated in secondary methylcellulose cultures in the presence of G418. IT is expected that the number of colony forming progenitors in the MSCV/neo population should mimic those from untransduced cells, while cells that express MSCV GATA-1s isoform may continue to form colonies through a significant number of more passages. In contrast, cells derived from the GATA-1 knock-down mice with trisomy 16, engineered to express wild-type GATA-1 may exhibit wild-type growth properties.

An alternative to utilizing bone marrow with trisomy 16 for the above retroviral studies is to infect GATA-1 knock-down progenitors directly with retroviruses that express RUNX1, which is a strong candidate for being involved in DS-AMKL.

The in vitro studies outlined above may provide very useful insights into the mechanisms by which GATA-1 mutations and trisomy 21 cooperate in leukemogenesis. In addition to the above in vitro exploitations, the recipient mice also may be lethally irradiated, and then their recipient bone marrow reconstituted with the bone marrow progenitors. This will be useful to do if immortalization is not detected in vitro. Furthermore, even if there is an increase in self-renewal of megakaryocyte progenitors, these cells should be assayed to determine if they can promote leukemogenesis in vivo.

In an exemplary protocol for the above in vivo determinations, wild-type female littermates (generated by the breeding scheme of FIG. 6) will be lethally irradiated to serve as recipients in the transplant studies. First, the mice will be subjected to 800 rads, then following a three hour break, they will be exposed to another 400 rads. Immediately after the second exposure, the marrow of the mice is reconstituted with bone marrow cells via retroorbital or tail vein injection. In this aspect, 10,000 bone marrow cells transduced with MSCV/neo-Tyr63Stop GATA-1, or MSCV/neo, are injected along with 100,000 normal isogenic cells from wild-type females of the same genetic background, to ensure radioprotection. This in vivo may be performed using previously reported protocols. For example, it has previously been demonstrated that overexpression of MLL-ELL fusion protein could immortalize myeloid precursors and lead to leukemia in recipient mice (Lavau, C., Luo, R. T., Du, C. and Thirman, M. J. (2000) Proc Natl Acad Sci USA, 97(20), 10984-9). Similar studies are contemplated using the GATA-1 constructs of the present invention.

The following six GATA-1 transplants, each into 5 recipient animals are specifically contemplated, however, given the teachings of the present invention those of skill in the art may perform similar protocols using other GATA-1 constructs:

| | | |
|---|---|---|
| 1. | MSCV-neo | GATA-1 knock-down progenitors |
| 2. | MSCV-neo, | GATA-1 knock-down, trisomy 16 |
| | | progenitors |
| 3. | MSCV-neo | wild-type GATA-1, trisomy 16 |
| | | progenitors |
| 4. | MSCV-neo | wild-type progenitors |
| 5. | MSCV/neo- Tyr63Stop | GATA-1 knock-down progenitors |
| | GATA-1 | |
| 6. | MSCV/neo- Tyr63Stop | GATA-1 knock-down, trisomy 16 |
| | GATA-1 | progenitors |
| 7. | MSCV/neo- Tyr63Stop | wild-type GATA-1, trisomy 16 |
| | GATA-1 | progenitors |
| 8. | MSCV/neo- Tyr63Stop | wild-type progenitors |
| | GATA-1 | |

In each case, male progenitor cells are used for ease of identifying the transplanted cells in female recipients. Furthermore, the transplanted cells will also be marked by virtue of their neomycin resistance. Following transplantation, mice will be monitored for six months for abnormal hematopoietic cell development. For example, the recipient mice will be weighed twice a week, and the animal's mobility, ability to reach food and water, and grooming habits. In addition, the mice will be phlebotomized every two months to monitor the CBC, and also to analyze stained blood smears for the presence of abnormal blasts in the peripheral blood. If the mice exhibit blasts in their blood, the cells will be isolated to determine whether the cells are megakaryoblastic or otherwise.

The present application also contemplates the generation and use of mice with a human DS-AMKL GATA-1 mutation and development of a mouse model of DS-AMKL. DNA samples from every patient with DS-AMKL harbor a frame-shift mutation within exon 2 of GATA-1 that block expression of full length GATA-1, but allow for production of the shortened, N-terminal deleted protein GATA-1s. Thus, while it is formally possible that DS-AMKL arises from the loss of full length GATA-1 and GATA-1s is an innocent bystander, GATA-1s may play a key role in the leukemic transformation. To study these properties of GATA-1s further, mice that harbor tandem loxP recombination sites surrounding exon 2 of GATA-1 will be generated. These mice will be different from the GATA-1 knock-down mice in that, upon induction of Cre recombinase, the short isoform of GATA-1 will be generated in place of full length GATA-1: a situation which more closely mimics DS-AMKL.

Using these mice, it will be possible to determine whether this alteration of GATA-1 alone can lead to the onset of AMKL, myelodysplastic syndrome, or abnormal growth of mutant GATA-1 progenitors. Separately, these studies will help in determining whether the short isoform can substitute for full length GATA-1 in certain lineages. Further studies will include breeding these novel GATA-1 mutant mice to mice with the murine equivalent of Down syndrome and to mice of the BXH-2 strain, which are predisposed to developing myeloid leukemias. These compound mutant mice will likely develop megakaryoblastic leukemia. This mouse model can then be used in identifying factors that cooperate with the GATA-1 mutations to promote leukemia. Since approximately 5% of AMLs and 30% of pediatric ALLs (hyperdiploid ALLs) have an acquired trisomy 21 (Heim, S. and Mitelman, F. (1995) Cancer Cytogenetics, 2nd Ed., Wiley-Liss, New York; Raimondi, S. C., Pui, C. H., Hancock, M. L., Behm, F. G., Filatov, L. and Rivera, G. K. (1996) Leukemia, 10(2), 213-24; Le Beau, M. M. and Larson, R. A. (1998) Hematology: Basic Principles and Practices, 3rd Ed. Cytogenetics and Neoplasia (Hoffman, R., Shattil, S. J., Furie, B., Cohen, H. J., Silberstein, L. E., and McGlave, P., Eds.), Churchill Livingstone, New York), this mouse model may be very useful in understanding the pathology of not only leukemias of Down syndrome, but also in a much large number of cancers.

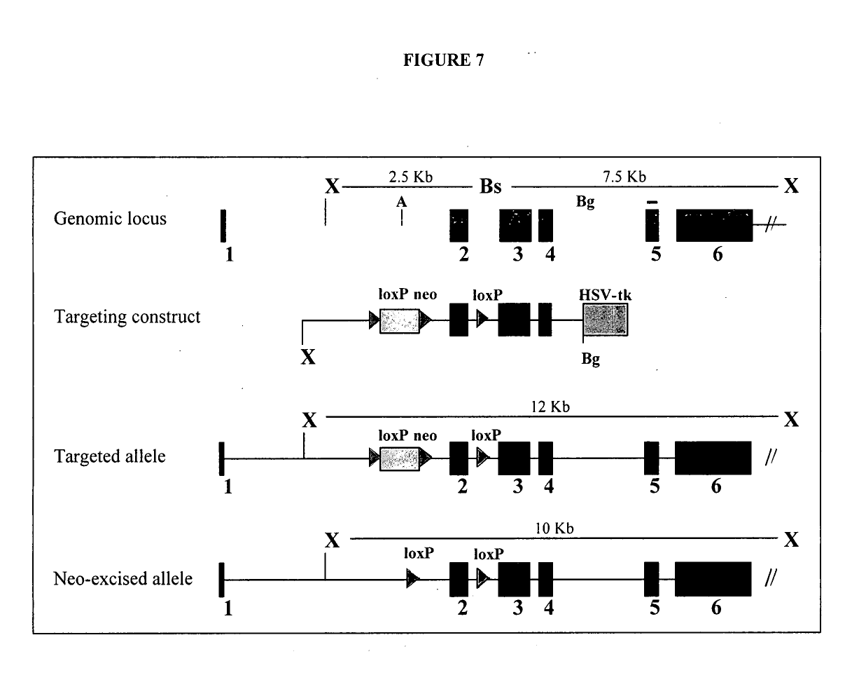

Previously homologous recombination has been used to introduce a specific point mutation in the Gata-1 gene in mice (Chang, A. N., Cantor, A. B., Fujiwara, Y., Lodish, M. B., Droho, S., Crispino, J. D. and Orkin, S. H. (2002) Proc Natl Acad Sci USA, 99(14), 9237-42). This missense mutation converted Val205 to Glycine, resulting in expression of a GATA-1 molecule that could not interact with its cofactor FOG-1. As a consequence of this alteration, hemizygous mutant males died during embryogenesis of anemia. Female mice heterozygous for the V205G mutation were often born with mild anemia that resolved, but were mildly thrombocytopenic during their life-span. A very similar strategy will be used to generate mice that will conditionally express the short isoform of GATA-1 in place of full length GATA-1. To mimic the situation that develops in human DS-AMKL blasts, the GATA-1 locus is conditionally targeted by floxing the second exon of GATA-1 with loxP recombination sites. FIG. 7 illustrates the targeting strategy that will be utilized. The GATA-1 murine genomic sequences are modified by introducing a floxed pGK-neomycin resistance cassette in the unique Avr II restriction site upstream of exon 2, and concomitantly position a single loxP sequence in a unique Bsa I restriction site downstream of exon 2. The targeting construct will be introduced into wild-type CJ7 ES cells by electroporation and select for clones resistant to 300 μg/ml G418.

Once the correctly targeted clones are identified, and it has been verified that the clones have a normal chromosome complement by cytogenetic analysis, the inventors will transiently express Cre recombinase by electroporating the ES cells with the pMC-Cre plasmid (Torres, R. M. and Kuhn, R. (1997) Laboratory Protocols for Conditional Gene Targeting, Oxford University Press, New York). The ES clones that have lost the neomycin cassette, but retain the two tandem loxP sites flanking exon 2 are screened. This is a standard means to remove the selectable marker (Torres, R. M. and Kuhn, R. (1997) Laboratory Protocols for Conditional Gene Targeting, Oxford University Press, New York). There are two reasons to remove the neomycin cassette. First, the presence of a pGK driven neomycin gene often alters expression of the gene into which it is integrated. Second, by leaving only two loxP sites within the gene, the probability that the two loxP sites flanking exon 2 will recombine in adult mice following Cre expression is increased.

Karyotyping is again performed to verify that the ES cells are karyotypically normal. This is followed by another control before using the ES cells for generating transgenic mice. Since exon 2 from the locus is being specifically excised, it will be desirable to determine that the removal of this exon does not have any adverse effects on the stability or splicing of the messenger RNA. GATA-1 mRNA is initiated in the first exon, and the third exon has a functional translation initiation codon; therefore, GATA-1s will be efficiently translated when exon 2 is removed. To confirm that GATA-1 mRNA is produced in these targeted ES cells even in the absence of exon 2, the control will again introduce Cre recombinase into these ES cells. The ES cells are then assayed for excision of exon 2 of GATA-1 by Southern blot analysis. Further, RT-PCR may be performed to assess whether GATA-1 mRNA that lacks exon 2 sequences is produced. Since GATA-1 is not expressed in undifferentiated ES cells, it may be necessary to culture the ES cells in methylcellulose for several days to generate embryoid bodies prior to the RT-PCR analysis.

Once it is confirmed that GATA-1 mRNA lacking exon 2 is produced by the conditional allele, the transgenic mice may be generated. To generate chimeras, the targeted ES cell clones are injected into wild-type C57B1/6 blastocysts, in conjunction with the University of Chicago Transgenic Core Facility. Male chimeras will then be mated to wild-type C57B1/6 female mice to generate heterozygous pups. Both Southern blot analysis and PCR assays can be performed to genotype mice for the loxP insertions and ensure that germ line transmission has occurred.

Mice that harbor this floxed allele of Gata-1 will likely not exhibit any abnormal phenotype. Once this has been verified, these mice are bred to mice that are transgenic for the Cre recombinase gene driven off the interferon inducible MX1 promoter (Kuhn, R., Schwenk, F., Aguet, M: and Rajewsky, K. (1995) Science, 269(5229), 1427-9; Kuhn, R. and Torres, R. M. (2002) Methods Mol Biol, 180, 175-204). MX1-Cre mice are commercially available from Jackson labs and are widely used for inducible excision of genes. For example, Higuchi and coworkers recently expressed AML1-ETO in a conditional manner, utilizing a MX1-Cre strategy (Higuchi, M., O'Brien, D., Kumaravelu, P., Lenny, N., Yeoh, E. J. and Downing, J. R. (2002) Cancer Cell, 1(1), 63-74). The MX1 promoter can be activated by treatment of mice with the synthetic double-stranded RNA, polyinosinic-polycytidylic acid (pI-pC; 250 μg per injection). This treatment results in activation of the promoter and expression of Cre recombinase in a broad range of tissues, but yields especially strong expression in sites of hematopoiesis including the bone marrow. Since GATA-1 is primarily restricted to hematopoietic cells, the knock-in would essentially exhibit lineage specificity. A preferred colony of mice is one which contains mice that harbor floxed alleles of Gata-1 that are also transgenic for MX1-Cre. To induce expression of Cre recombinase, and the resulting excision of exon 2 from Gata-1, mice are treated with three intraperitoneal injections of pI-pC over a six-day period. The following mice when generated will be followed for the development of disease:

| | |
|---|---|
| 1. | GATA-1 E2 floxed mice, no MX1-Cre, |
| 2. | GATA-1 E2 floxed mice, no MX1-Cre, + pI-pC, |
| 3. | GATA-1 E2 floxed mice, with MX1 Cre transgene, |
| 4. | GATA-1 E2 floxed mice, with MX1 Cre transgene + pI-pC |
| 5. | Wild-type GATA-1 mice, with MX1-Cre transgene. |
| 6. | Wild-type GATA-1 mice, with MX1-Cre transgene + pI-pC. |

These mice will be tested for development of myelodysplasia, thrombocytopenia and/or leukemia. At weaning, the mice are tail clipped for genotyping purposes and also ear tagged for identification. In addition, 100 μl peripheral blood is harvested from the retroorbital sinus for both karyotype/FISH analysis and for establishing the baseline hematologic values. With time, the mice will be closely monitored for signs of development of leukemia. This will include weighing each mouse twice a week, and observing its mobility, ability to reach food and water, and grooming habits. In addition, the mice will be phlebotomized two weeks following the pI-pC injection, and every two months after, to monitor the CBC, and also to analyze stained blood smears for the presence of abnormal blasts in the peripheral blood.

To characterize the genetic features of these leukemias, cytogenetic and spectral karyotyping (SKY) analysis is performed. Bone marrow and spleen cells from mice with leukemia will be used to prepare metaphase cells following short-term (24-72 hr) cultures as described previously (Roulston, D. and Le Beau, M. M. (1997) Cytogenetics Analysis of Hematologic Malignant Diseases, 3rd Ed. The AGT Cytogenetics Laboratory Manual (Barch, M. J., Knutsen, T., and Spurbeck, J., Eds.), Lippincott-Raven, Philadelphia). The chromosomes are stained using a trypsin-Giemsa banding technique (Roulston, D. and Le Beau, M. M. (1997) Cytogenetics Analysis of Hematologic Malignant Diseases, 3rd Ed. The AGT Cytogenetics Laboratory Manual (Barch, M. J., Knutsen, T., and Spurbeck, J., Eds.), Lippincott-Raven, Philadelphia), and chromosomes are identified according to the standardized mouse karyotype as refined by Cowell (Cowell, J. K. (1984) Chromosoma, 89, 294-320). SKY will be performed using the Applied Spectral Imaging (ASI) SkyPaint™ kit for mouse chromosomes and SkyPaint™ detection reagents as described previously (Le Beau, M. M., Bitts, S., Davis, E. M. and Kogan, S. C. (2002) Blood, 99(8), 2985-91). A minimum of ten metaphase cells are examined using each technique (cytogenetic and SKY analysis) per mouse leukemia.

There is a chance that, upon excision of exon 2, GATA-1 mRNA will not splice correctly or will be degraded. In this event, a conditional knock-out of GATA-1 is created. This may be a useful tool to study the requirements for GATA-1 during adult hematopoiesis.

As an alternative, a GATA-1 genomic targeting vector has been created that contains a four base pair insertion immediately following the Tyr63 codon within exon 2 of GATA-1. This insertion is the same as that detected in the malignant cells of patient DS-AMKL-1. As a consequence of this insertion, these mutant alleles failed to express full length GATA-1, but continued to produce GATA-1s protein. In addition to this insertion, a silent change in a nearby residue is also introduced, which results in the introduction of a new restriction site, for ease in genotyping both ES clones and mice. The targeting construct also harbors a floxed pGK-neomycin resistance cassette in the intron downstream. This DNA has been electroporated into CJ7 ES cells and clones that developed resistance to 300 μg/ml G418 were selected. The karyotypically normal clones from these studies will be induced to transiently express Cre recombinase in the ES cells to delete the neomycin selection cassette. To produce chimeras, the targeted ES cell clones are injected into wild-type C57B1/6 blastocysts. Male chimeras will then be mated to wild-type C57B1/6 female mice to generate heterozygous pups. Southern blot analysis and PCR assays can be used to genotype mice for the GATA-1 mutations and ensure that germ line transmission has occurred. These mice resulting from these manipulations will have a constitutional defect in GATA-1.

Since previous reports have shown that GATA-1s could not substitute for full length GATA-1 in vivo unless it was expressed at very high levels (Shimizu, R., Takahashi, S., Ohneda, K., Engel, J. D. and Yamamoto, M. (2001) Embo J, 20(18), 5250-60), it is likely that the homozygous females, and hemizygous males, whose only allele of GATA-1 is mutated, will die in embryogenesis. Also, it is believed that heterozygous females will be born at the normal frequency, but may exhibit a phenotype similar to GATA-1 knock-down and GATA-1 V205G mice, i.e., a mild thrombocytopenia with accumulation of abnormal megakaryocytes in the spleen and bone marrow. These novel GATA-1 Tyr63 knock-in mutant mice are different from the previous models of GATA-1 deficiency in that approximately half of their hematopoietic progenitors will express the short isoform of GATA-1 s in the absence of full length GATA-1. These females are DS-AMKL models. The major drawback to this model is that the males will most likely not be viable, which will make the breeding to DS mice more challenging. Since male Ts65Dn mice are sterile, the novel DS-AMKL models cannot be cross-bred with Ts65Dn males, however, the DS-AMKL may be crossed with the Ts1Cje model.

Using the teachings provided herein, those of skill in the art will be able to identify cooperating factors in DS-AMKL leukemogenesis. Development of leukemia in the GATA-1 E2 floxed, MX1-Cre mice may be a rare event. To induce leukemogenesis, these mutants may be crossed into two different strains of mice. Firstly, the mutants may be crossed with the Down syndrome mouse by breeding GATA-1 E2 floxed, MX1-Cre mice to the Ts65Dn Down syndrome mice, in a manner similar to that described in FIG. 6. Secondly, the mutants may be crossed with BXH-2 mice yields an unbiased approach to identify the genes that cooperate with GATA-1 mutations in the progression of DS-AMKL.

Retroviral insertional mutagenesis can be used to identify genes that cooperate in tumorigenesis. Retroviral insertions in the genome can transform cells by inactivating tumor suppressor genes or activating proto-oncogenes. In recent studies, Dr. Anton Berns infected donor bone marrow progenitors with MuLV retroviruses, and then followed recipient mice for the development of leukemia or lymphoma (Selten, G., Cuypers, H. T., Zijlstra, M., Melief, C. and Berns, A. (1984) Embo J, 3(13), 3215-22; van Lohuizen, M., Verbeek, S., Krimpenfort, P., Domen, J., Saris, C., Radaszkiewicz, T. and Berns, A. (1989) Cell, 56(4), 673-82; Jonkers, J. and Berns, A. (1996) Biochim Biophys Acta, 1287(1), 29-57). Similar experiments also have been performed in mutant mouse strains (Berns, A., Mikkers, H., Krimpenfort, P., Allen, J., Scheijen, B. and Jonkers, J. (1999) Cancer Res, 59(7 Suppl), 1773s-1777s). Alternatively, other groups have used a genome wide approach of retroviral insertional mutagenesis to identify cancer causing genes. For example, Lund et al. identified genes that promote tumorigenesis in Cdkn2a-deficient mice (Lund, A. H., Turner, G., Trubetskoy, A., Verhoeven, E., Wientjens, E., Hulsman, D., Russell, R., DePinho, R. A., Lenz, J. and Van Lohuizen, M. (2002) Nat Genet, 32(1), 160-5). Furthermore, Mikkers et al. recently used high-throughput retroviral tagging to identify factors that could promote leukemia in Myc transgenic mice that lacked Pim1 and Pim2 (Mikkers, H., Allen, J., Knipscheer, P., Romeyn, L., Hart, A., Vink, E. and Berns, A. (2002) Nat Genet, 32(1), 153-9).

The BXH-2 inbred strain of mice provides a very powerful in vivo way to identify new genes involved in cancer (Suzuki, T., Shen, H., Akagi, K., Morse, H. C., Malley, J. D., Naiman, D. Q., Jenkins, N. A. and Copeland, N. G. (2002) Nat Genet, 32(1), 166-74). These mice harbor an ecotropic murine leukemia virus, which is passed from one generation to the next by horizontal transmission and acts as an insertional mutagen. As a consequence, greater than 90% BXH-2 mice develop leukemia within 1 year (Li, J., Shen, H., Himmel, K. L., Dupuy, A. J., Largaespada, D. A., Nakamura, T., Shaughnessy, J. D., Jr., Jenkins, N. A. and Copeland, N. G. (1999) Nat Genet, 23(3), 348-53). The vast majority of leukemias that occur in BXH-2 mice are acute myeloid leukemias, although B and T-cell leukemias are also observed. Disease genes can be identified by cloning the common viral integration sites in the BXH-2 leukemias using inverse PCR. BXH-2 mice can be exploited in screens to identify genes that cooperate with genetic defects during progression of acute leukemias. For example, Largaespada et al. transferred an Nf-1 mutant allele into the BXH-2 background by repeated backcross matings (Largaespada, D. A., Brannan, C. I., Jenkins, N. A. and Copeland, N. G. (1996) Nat Genet, 12(2), 137-43). BXH-2 mice with the Nf-1 mutation developed AML faster that wild-type BXH-2 mice. The inactivation of tumor suppressor genes or the activation of proto-oncogenes unlinked to Nf-1 by the proviral integrations within BXH-2 strain are believed to be responsible for the accelerated leukemogenesis.

To cross GATA-1 E2 floxed, MX1-Cre mice into the BXH-2 strain, homozygous female GATA-1 E2 floxed, MX1-Cre transgenic female mice are crossed to male BXH-2 mice. The F1 litter will be comprised of hemizygous males and heterozygous females, with 50% harboring the MX1-Cre transgene. This strain is then inbred for at least 3 generations. During this breeding process, the mice are monitored for development of leukemia. These mice will develop leukemia within one year, but in the absence of MX1-Cre induction, the mice will develop primarily myeloid leukemias. Once third generation pups that harbor the GATA-1 E2 floxed mutation and the MX1-Cre transgene are generated, MX1-Cre activity is induced by injection of pI-pC, immediately after weaning. These latter mice, will develop megakaryoblastic leukemia. When evidence of this malignancy is detected, bone marrow is harvested and the cells are cultured in vitro and analysed for the viral integration site.

By comparing the leukemias found in GATA-1 E2 floxed, MX1-Cre-BXH-2 mice that have received pI-pC treatment with identical mice that have not received pI-pC, the characteristics of megakaryoblastic leukemia can be assessed in these model mice. Once megakaryoblastic leukemia is identified, an inverse PCR approach can be used to clone the viral integration sites (Li, J., Shen, H., Himmel, K. L., Dupuy, A. J., Largaespada, D. A., Nakamura, T., Shaughnessy, J. D., Jr., Jenkins, N. A. and Copeland, N. G. (1999) Nat Genet, 23(3), 348-53). Briefly, DNA from the leukemic cells will be digested with a restriction enzyme that cleaves once within the provirus, and the linear DNA will then be ligated to form circular DNA. Next, the circular DNA will be amplified by PCR using primers against the proviral DNA sequences, and amplified in a second round with nested primers that harbor dUMP tails to facilitate cloning into a plasmid pAMP1. Finally, sequencing primers homologous to the cloning site will be used to sequence 600-700 base pairs of cellular DNA from each end of the insert. The availability of genome sequence information for the mouse permits unambiguous map assignment of the short genomic DNA fragments generated by this method. Those of skill in the art have previously characterized a number of proviral insertion sites. For example, Dr. David Largaespada (Univ. of Minnesota) has isolated >60 new proviral insertion sites near CpG islands and a number of new candidate myeloid leukemia genes from BXH-2 AMLs, and has developed a Web-based BLAST server that can be used to query proviral insertion site sequences (http://www.cancer.umn.edu/blast/). Dr. Neal Copeland has also generated a website containing data on hundreds of integration sites in the BXH-2 murine model for myeloid leukemias (http://genome2.ncifcrf.gov/VIMDB). Contrasting the integration sites identified in the present invention with those in these databases will be a powerful approach to identify common integration sites, and for prioritizing which genes are chosen for further analysis. Genes that reside on mouse chromosome 16 will be especially interesting candidates, since they likely are involved in Down syndrome.

The megakaryoblastic leukemias identified from the mice generated herein will also be subjected to cytogenetic analysis. In previous studies, spectral karyotyping analysis has been used to examine leukemias arising in murine models of the recurring chromosomal translocations in AML. For example, it was demonstrated that murine leukemias initiated by PML-RARA have a defined spectrum of genetic changes, and that these secondary changes recapitulate, in part, the cytogenetic abnormalities found in human acute promyelocytic leukemia (Le Beau, M. M., Bitts, S., Davis, E. M. and Kogan, S. C. (2002) Blood, 99(8), 2985-91). To identify cooperating cytogenetic abnormalities in the leukemias arising in the GATA-1 E2 floxed-Ts65Dn Down syndrome mice and the GATA-1 E2 floxed—BXH-2 mice, SKY analysis will be performed leukemias in each genetic subgroup. The characterization of the karyotypic pattern of murine leukemias will allow identification of mutations contributing or cooperating in leukemogenesis; identification of chromosomal bands containing the involved genes; comparison of the involved regions with the syntenic regions in human chromosomes to contrast the chromosomal abnormalities observed in patients with DS and AML; and correlation of the morphologic, immunophenotypic and cytogenetic features.

C. Diagnostic Methods

The present invention provides methods of diagnosing transient myeloproliferative disorder (TMD) and other acute megakaryoblastic leukemia by providing a tissue sample from a person suspected of having such a disorder, and determining the loss or mutation of a GATA-1 encoding nucleic acid in the cells of said tissue. In particular, this aspect of the invention provides a method of diagnosing such a condition comprising the following steps: (a) contacting a cell sample nucleic acid with a microarray discussed herein under conditions suitable for hybridization; (b) providing hybridization conditions suitable for hybrid formation between the cell sample nucleic acid and a polynucleotide of the microarray; (c) detecting the hybridization; and (d) diagnosing the disorder condition based on the results of detecting the hybridization. In preferred embodiments, the sample is selected from the group consisting of blood, an amniocentesis sample, somatically in utero fetal blood, and bone marrow.

Suitable hybridization conditions for the diagnostic methods are those conditions that allow the detection of gene expression from identifiable expression units such as genes. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

The sequence of the GATA-1 cDNA can be used to generate probes to detect chromosome abnormalities in the GATA-1. These probes may be generated from both the sense and antisense strands of double-stranded DNA. The term “GATA-1 probe” refers to both genomic and cDNA probes derived from the GATA-1 gene.

cDNA probes capable of detecting mutations in exon 2 of GATA-1 gene are particularly preferred. Part or all of the GATA-1 cDNA sequence may be used to create a probe capable of detecting aberrant transcripts of GATA-1 which lack part or all of exon 2.

The GATA-1 nucleic acid sequences provided in SEQ ID NO:1 and 2 (for wild-type GATA-1) and SEQ ID NO: 4-10, 12, 14, 16 and 18 can be used to create probes to detect mutations in the GATA-1 gene that lead to AMKL. Using the probes of the present invention, several methods are available for detecting chromosome abnormalities in the GATA-1 gene. Such methods include, for example, Polymerase Chain Reaction (PCR) technology, restriction fragment length analysis, and oligonucleotide hybridization using, for example, Southern and Northern blotting and in situ hybridization.

PCR technology is practiced routinely by those having ordinary skill in the art and its uses in diagnostics are well known and accepted. Methods for practicing PCR technology are disclosed in PCR Protocols: A Guide to Methods and Applications, Innis, M. A. et al., Eds., Academic Press, San Diego, Calif. 1990, and RT-PCR, Clontech Laboratories (1991), which are incorporated herein by reference. Applications of PCR technology are disclosed in Polymerase Chain Reaction, Erlich, H A. et al., Eds., Cold Spring Harbor Press, Cold Spring Harbor, N.Y. 1989, which is incorporated herein by reference.

PCR technology allows for the rapid generation of multiple copies of DNA sequences by providing 5′ and 3′ primers that hybridize to sequences present in a DNA molecule, and further providing free nucleotides and an enzyme which fills in the complementary bases to the DNA sequence between the primers with the free nucleotides to produce a complementary strand of DNA. The enzyme will fill in the complementary sequences between probes only if both the 5′ primer and 3′ primer hybridize to DNA sequences on the same strand of DNA.

RNA is isolated from hematopoietic cells of a person suspected of having AMKL, and cDNA is generated from the mRNA. If the cDNA of the chimeric ALL-1/AF-4 gene is present, both primers will hybridize to the cDNA and the intervening sequence will be amplified. The PCR technology therefore provides a straightforward and reliable method of detecting the chimeric gene.

According to the invention, diagnostic kits can be assembled which are useful to practice oligonucleotide hybridization methods of distinguishing abnormalities in GATA-1. Such diagnostic kits comprise a labelled oligonucleotide which hybridizes, for example, to the mutant GATA-1 that lacks all or part of exon 2, or has a mutation in exon 2 but which does not hybridize to nucleic acid transcripts not associated with aberrations. Accordingly, diagnostic kits of the present invention comprise, for example, a labelled probe that includes GATA-1 mutants having a mutation in exon 2 selected from the group consisting of SEQ ID NO:5, SEQ ID NO:6, SEQ ID NO:7, SEQ ID NO:8, SEQ ID NO:9, SEQ ID NO:12, SEQ ID NO:14, SEQ ID NO:16, and SEQ ID NO:18. Of course, the diagnostic kits of the invention also are contemplated to include labelled probes derived from the full-length wild-type sequence of SEQ ID NO:1 and SEQ ID NO:2, or from wild-type exon 2 found in SEQ ID NO:4. It is contemplated that exemplary probes may be 8, 9, 10, 11, 12, 13, 14, 15, 16, 17, 18, 19, 20, 25, 30, 35, 40, 45, 50, 55, 60, 65, 70, 75, 80, 85, 90, 95, 100 or more contiguous base pairs from the above sequences will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

It is preferred that labelled probes of the oligonucleotide diagnostic kits according to the present invention are labelled with a radionucleotide. The oligonucleotide hybridization-based diagnostic kits according to the invention preferably comprise DNA samples that represent positive and negative controls. A positive control DNA sample is one that comprises a nucleic acid molecule which has a nucleotide sequence that is fully complementary to the probes of the kit such that the probes will hybridize to the molecule under assay conditions. A negative control DNA sample is one that comprises at least one nucleic acid molecule, the nucleotide sequence of which is partially complementary to the sequences of the probe of the kit. Under assay conditions, the probe will not hybridize to the negative control DNA sample.

Probes useful as diagnostics can be used not only to diagnose the onset of illness in a patient, but may also be used to assess the status of a patient who may or may not be in remission. It is believed that emergence of a patient from remission is characterized by the presence of cells containing chromosome abnormalities. Thus, patients believed to be in remission may be monitored using the probes of the invention to determine their status regarding progression or remission from disease. Use of such probes will thus provide a highly sensitive assay the results of which may be used by physicians in their overall assessment and management of the patient's illness.

Antisense oligonucleotides which hybridize to at least a portion of an aberrant transcript resulting from a mutation of the GATA-1 gene are also contemplated by the present invention. The oligonucleotide may match the target region exactly or may contain several mismatches. Thus, molecules which bind competitively to RNA coded by, for example, the GATA-1 gene, for example, are envisioned for therapeutics.

The term “oligonucleotide” as used herein includes both ribonucleotides and deoxyribonucleotides, and includes molecules which may be long enough to be termed “polynucleotides.” Oligodeoxyribonucleotides are preferred since oligoribonucleotides are more susceptible to enzymatic attack by ribonucleotides than deoxyribonucleotides. It will also be understood that the bases, sugars or internucleotide linkages may be chemically modified by methods known in the art. Modifications may be made, for example, to improve stability and/or lipid solubility. For instance, it is known that enhanced lipid solubility and/or resistance to nuclease digestion results by substituting a methyl group or sulfur atom for a phosphate oxygen in the internucleotide phosphodiester linkage. The phosphorothioates, in particular, are stable to nuclease cleavage and soluble in lipid. Modified oligonucleotides are termed “derivatives.”

The oligonucleotides of the present invention may be synthesized by any of the known chemical oligonucleotide synthesis methods. See for example, Gait, M. J., ed. (1984), Oligonucleotide Synthesis (IRL, Oxford). Since the entire sequence of the GATA-1 gene is known along with partial sequences of the GATA-1 gene, antisense oligonucleotides hybridizable with any portion of these sequences may be prepared by the synthetic methods known by those skilled in the art.

In certain embodiments, the 10 or more oligonucleotide probes may be arrayed in the form of a diagnostic chip or “microarray” for the analysis and expression of these genes in various cell types. Such a microarray could be used for measuring gene expression of GATA-1 and preferably comprises distinct sequences derived from wild-type and mutant GATA-1. Methods of making such microarrays are discussed in detail elsewhere in the specification.

D. Nucleic Acids of the Present Invention and Methods of Achieving their Recombinant Expression in Cells

The present invention is directed to methods and compositions for exploiting the finding that aberrations in the expression of the GATA-1 gene, and more particularly, mutations of exon 2 of said gene are indicative and predispose an individual to AMKL, and TMD. Any reference to a nucleic acid of the present invention should be understood as encompassing a vector comprising that polynucleotide and a host cell containing that vector or nucleic acid and, in some cases, capable of expressing the protein product of that nucleic acid. Cells expressing nucleic acids of the present invention will be useful in certain diagnostic and screening situations, and methods of making and using such cells are described below.

The nucleic acid sequences disclosed in SEQ ID NO:1, 2, 4-10, 12, 14, 16 and 18 encode various portions of GATA-1. The sequence for GATA-1 is well known to those of skill in the art and genomic DNA, cDNA, mRNA, as well as recombinant and synthetic sequences and partially synthetic sequences derived therefrom which may encode an entire protein, polypeptide, or allelic variant thereof can be used in the present invention.

Nucleic acids having sequences corresponding to a GATA-1 wild-type sequence such as that seen in SEQ ID NO:1 may be obtained from genomic DNA, i.e., cloned directly from human cells. However, the nucleic acid also could be obtained from complementary DNA (cDNA). Also contemplated is a cDNA plus a natural intron or an intron derived from another gene; such engineered molecules are sometime referred to as “mini-genes.”

The term “cDNA” is intended to refer to DNA prepared using messenger RNA (mRNA) as a template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are targets in antisense methods of modulating gene expression.

It may be advantageous to combine portions of genomic DNA with cDNA or synthetic sequences to generate specific constructs. For example, where an intron is desired in the ultimate construct, a genomic clone is suitable. The cDNA or a synthesized polynucleotide may provide more convenient restriction sites for the remaining portion of the construct and, therefore, would be used for the rest of the sequence. In particularly preferred aspects, it may be advantageous to prepare variants of the 220 nucleotide base sequence of SEQ ID NO:4 to produce GATA-1 derivatives in which exon 2 has been mutated. SEQ ID NOs:4, 5, 6, 7, 8 and 9 are examples of naturally occurring mutations in exon 2. These mutations introduce stop codons to shorten the expression product of exon 2 of GATA-1, as can be seen from SEQ ID NOs:11, 13, 15, 17, and 19. Given these findings other mutations may be engineered using site-directed mutagenesis to produce shortened or aberrant expression of GATA-1.

As used in this application, the term “functionally equivalent codon” is used herein to refer to codons that encode the same amino acid, such as the six codons for arginine or serine, and also refers to codons that encode biologically equivalent amino acids, as discussed in the following pages.

CODON TABLE

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The present invention also encompasses DNA segments that are complementary, or essentially complementary, to the sequences set forth in any one of sequences of SEQ ID NO:1. Nucleic acid sequences that are “complementary” are those that are capable of base-pairing according to the standard Watson-Crick complementarity rules. That is, guanidylate (deoxyguanidylate) pairs with cytidylate (deoxycytidylate) and adenylate pairs with uridylate (thymidylate). Inclusion of less common bases such as inosine, 5-methylcytosine, 6-methyladenine, hypoxanthine and others in hybridizing sequences does not interfere with pairing, and is contemplated as falling within the scope of the invention.

As used herein, the term “complementary sequences” means nucleic acid sequences that are substantially complementary, as may be assessed by the same nucleotide comparison set forth above, or as defined as being capable of hybridizing to a nucleic acid having a sequence of SEQ ID NO:1 under stringent conditions such as those described herein. Those of skill in the art will understand what is meant by stringent conditions and are referred to page 11.45 of Molecular Cloning: A laboratory Manual, 2nd Ed. Cold Spring Harbor Laboratory Press, Cold Spring Harbor, N.Y., or the conditions set forth above for diagnostic purposes.

The hybridizing nucleic acids may be shorter (i.e., oligonucleotides or probes). Sequences of about 17 bases long should occur only once in the human genome and, therefore, should suffice to specify a unique target sequence. Nucleotide sequences of this size that specifically hybridize to any of the nucleic acids sequences of SEQ ID NO:1, SEQ ID NO:2, SEQ ID NO:4-10, SEQ ID NO:12, SEQ ID NO:14, and SEQ ID NO:18 are useful as probes or primers. As used herein, an oligonucleotide that “specifically hybridizes” to a nucleic acid of any of these sequences means that hybridization under suitably (e.g., high) stringent conditions allows discrimination of one or a few hybridizing sequences preferably one sequence, from other genes. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

Suitable hybridization conditions are well known to those of skill in the art. In certain applications, it is appreciated that lower stringency conditions may be required. Under these conditions, hybridization may occur even though the sequences of the interacting strands are not perfectly complementary, being mismatched at one or more positions. Conditions may be rendered less stringent by, e.g., increasing salt concentration and/or decreasing temperature.

One method of using probes and primers of the present invention is in the search for gene expression in human cells. Normally, the target DNA will be a genomic or cDNA library, although screening may involve analysis of RNA molecules. By varying the stringency of hybridization, and the region of the probe, different degrees of homology may result in hybridization.

Given the above disclosure of the nucleic acid constructs, it is possible to produce the gene product of any GATA-1 encoding gene or mutant thereof by routine recombinant DNA/RNA techniques. A variety of expression vector/host systems may be utilized to contain and express the coding sequence. These include, but are not limited to, microorganisms such as bacteria transformed with recombinant bacteriophage, plasmid, phagemid, or cosmid DNA expression vectors; yeast transformed with yeast expression vectors; insect cell systems infected with viral expression vectors (e.g., baculovirus); plant cell systems transfected with virus expression vectors (e.g., Cauliflower Mosaic Virus, CaMV; Tobacco Mosaic Virus, TMV) or transformed with bacterial expression vectors (e.g., Ti or pBR322 plasmid); or even animal cell systems. Mammalian cells that are useful in recombinant protein productions include, but are not limited to, VERO cells, HeLa cells, Chinese hamster ovary (CHO) cells, COS cells (such as COS-7), W138, BHK, HepG2, 3T3, RIN, MDCK, A549, PC12, K562 and HEK 293 cells.

In other embodiments, expression vectors may be used to introduce the genes of the present invention into host cells to produce recombinant cells. The invention contemplates expression via cassettes, which requires that appropriate signals or in various regulatory elements be provided in the vectors, such as enhancers, promoters, expression factor binding sites, and terminators, collectively controlling expression of the genes of interest in the host cells of interest.

Throughout this application, the term “expression construct” or “expression vector” is meant to include any type of genetic construct containing a nucleic acid coding for a gene product in which part or all of the nucleic acid encoding sequence is capable of being transcribed. The transcript may be translated into a protein and this process may be facilitated by inclusion of a ribosome binding site and/or a stop codon(s) in the expression vector, but it need not be. In certain embodiments, expression includes both transcription of a gene and translation of the cognate mRNA into a protein gene product.

The nucleic acid encoding a gene product is under transcriptional control of a promoter. A “promoter” refers to a DNA sequence recognized by the native synthetic machinery of the cell, or introduced synthetic machinery, required to initiate the specific transcription of a gene. The phrase “under transcriptional control” means that the promoter is in the correct location and orientation in relation to the coding region of interest to control RNA polymerase initiation and appropriate extension of the nascent mRNA corresponding to the gene.

The term promoter will be used herein to refer to a group of transcriptional control modules that are clustered around the initiation site for RNA polymerase II. Additional promoter elements regulate the frequency of transcriptional initiation. Typically, these are located in the region 30-110 bp upstream of the transcription start site, although a number of promoters have recently been shown to contain functional elements downstream of the start site as well, and constructs containing such promoters are contemplated by the invention. The spacing between promoter elements frequently is flexible, so that promoter function is preserved when elements are inverted or moved relative to one another. In the tk promoter, for example, the spacing between promoter elements can be increased to 50 bp before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the cell of interest. Thus, where a bacterial host cell is used, it is preferable to position the nucleic acid coding region adjacent to, and under the control of, a promoter that is capable of being expressed in a bacterial cell. Generally speaking, such a promoter is a bacterial or a phage promoter.

Suitable promoters for prokaryotes include, for example, the trp promoter (inducible by tryptophan deprivation), the lac promoter (inducible with the galactose analog IPTG), the β-lactamase promoter, and the lambda phage derived PL promoter (derepressible by temperature variation if the cIt5 marker is also used in the expression system). Other useful promoters include those for alpha-amylase, protease, Spo2, spac, and tac promoters. Especially preferred promoters include the kanamycin resistance promoter, GI 3, and the endogenous or native promoter for whichever gene is being introduced.

Promoters that may be used for expression in yeast include the 3-phospho-glycerate kinase promoter and those for other glycolytic enzymes, as well as promoters for alcohol dehydrogenase and yeast phosphatase. Also suited are the promoters for transcription elongation factor (TEF) and lactase. Mammalian expression systems generally may include the SV40 promoter known constitutive promoters functional in such cells, or regulatable promoters such as the metallothionein promoter, which is controlled by heavy metals or gluco-corticoid concentration.

All of the above promoters, well known and readily available to those of skill in the art, can be used to obtain high-level expression of the coding sequence of interest. The use of other viral or mammalian cellular, viral or bacteriophage promoters which are well-known in the art to achieve expression of a coding sequence of interest are contemplated as well, provided that the levels of expression are sufficient for a given purpose. By employing a promoter with well known properties, the level and pattern of expression of the protein of interest following transfection or transformation can be optimized.

Selection of a promoter that is regulated in response to a specific physiologic or synthetic signal(s) can permit inducible or derepressible (i.e., controllable) expression of the gene product. Several such promoter systems are available for production of viral vectors. One exemplary system is the ecdysone system (Invitrogen, Carlsbad, Calif.), which is designed to allow regulated expression of a gene of interest in mammalian cells. It consists of a tightly regulated expression mechanism that allows barely detectable basal level expression of a transgene, but over 200-fold inducibility of expression.

Translation control sequences include a ribosome binding site (RBS) in prokaryotic systems, whereas in eukaryotic systems translation may be controlled by a “TATA” box sequence which may also contain initiation codon such as AUG.

Another regulatory element contemplated for use in the present invention is an enhancer. These are genetic elements that increase, or enhance, transcription; enhancers may be located a considerable distance from a functionally related coding region (separation of several KB or more), the relative locations of enhancer and coding region is not specific (the enhancer may be 5′, 3′ or internal to the coding region), and the orientation of the enhancer itself is not specific (some enhancers function is inverted orientation). Promoters and enhancers are often overlapping and contiguous, often seeming to have a very similar modular organization. Enhancers useful in the present invention are well known to those of skill in the art and will depend on the particular expression system being employed (Scharf et al. Results Probl Cell Differ, 20, 125-62, 1994; Bittner et al., Methods in Enzymol, 15, 516-544, 1987).

There are a number of ways in which expression vectors may be introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genomes and express viral genes stably and efficiently have made them attractive candidates for the transfer of foreign genes into mammalian cells (Ridgeway, In: Rodriguez R L, Denhardt D T, eds. Vectors: A survey of molecular cloning vectors and their uses. Stoneham: Butterworth, pp. 467 492, 1988; Nicolas et al., In: Vectors: A survey of molecular cloning vectors and their uses, Rodriguez & Denhardt (eds.), Stoneham: Butterworth, pp. 493 513, 1988; Baichwal et al., In: Gene Transfer, Kucherlapati ed., New York, Plenum Press, pp. 117-148, 1986; Temin, In: gene Transfer, Kucherlapati (ed.), New York: Plenum Press, pp. 149 188, 1986).

Several non-viral methods for the transfer of expression constructs into cultured bacterial cells are contemplated by the present invention. This section provides a discussion of methods and compositions of non-viral gene transfer. DNA constructs of the present invention are generally delivered to a cell and, in certain situations, the nucleic acid or the protein to be transferred may be transferred using non-viral methods. The non viral methods include calcium phosphate precipitation (Graham et al., Virology, 52:456-467, 1973; Chen et al., Mol. Cell. Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. (USA), 76:3348-3352, 1979; Felgner, Sci Am. 276(6):102 6, 1997; Felgner, Hum Gene Ther. 7(15):1791 3, 1996), cell sonication (Fechheimer et al., Proc. Natl. Acad. Sci. (USA), 84:8463-8467, 1987), gene bombardment using high velocity microprojectiles (Yang et al., Proc. Natl. Acad. Sci USA, 87:9568-9572, 1990), conjugation (Gavigan et al. In: Tanya Parish and Neil G. Stoker (eds). Mycobacteria Protocols, pp. 119-128 1998. Humana Press, Twtowa, N.J.) and receptor-mediated transfection (Wu et al., J. Biol. Chem., 262:4429-4432, 1987; Wu et al., Biochemistry, 27:887-892, 1988; Wu et al., Adv. Drug Delivery Rev., 12:159-167, 1993).

The expression construct also may be entrapped in a liposome. (Ghosh et al., In: Liver diseases, targeted diagnosis and therapy using specific receptors and ligands, Wu et al. ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810 4, 1997).

Also contemplated in the present invention are various commercial approaches involving “lipofection” technology. In certain embodiments of the invention, the liposome may be complexed with a hemagglutinating virus (HVJ). This has been shown to facilitate fusion with the cell membrane and to promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991).

Other vector delivery systems which can be employed to deliver a nucleic acid encoding a given gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu et al., 1993, supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu et al., 1987, supra) and transferrin (Wagner et al., Proc. Natl. Acad. Sci. (USA), 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci. (USA) 91:4086-4090, 1994) and epidermal growth factor (EGF) has also been used to deliver genes to squamous carcinoma cells (Myers, EPO 0273085).

Another embodiment of the invention for transferring a naked DNA expression construct into cells may involve particle bombardment. This method depends on the ability to accelerate DNA coated microprojectiles to a high velocity, allowing them to pierce cell membranes and enter cells without killing them (Klein et. al., Nature, 327:70-73, 1987). Several devices for accelerating small particles have been developed. One such device relies on a high voltage discharge to generate an electrical current, which in turn provides the motive force (Yang et al., Proc. Natl. Acad. Sci. (USA), 87:9568-9572, 1990). The microprojectiles used to date have consisted of biologically inert substances such as tungsten or gold beads.

E. Polynucleotide Microarrays

As discussed above, microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays.

As discussed above, DNA-based microarrays provide a simple way to explore the expression of GATA-1 in samples from patients in a diagnostic context. Such microarrays also could be used to screen for new GATA-1 related sequences. In the present invention, least 10 oligonucleotides having distinct sequences derived from wild-type and mutant GATA-1 may be presented in a DNA microarray for the analysis and expression of these genes in various cell types. The sequences may be derived from any of the sequences set forth in SEQ ID NO:1, SEQ ID NO:2, SEQ ID NOs:4-10, SEQ ID NO:12, SEQ ID NO:14, SEQ ID NO:16 and SEQ ID NO:18 and include or any fragment thereof. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays. A microarray composition of the present invention can be employed for the diagnosis and treatment of any condition or disease in which the dysfunction or GATA-1 is implicated.

The term “microarray” refers to an ordered arrangement of hybridizable array elements. The array elements are arranged so that there are preferably at least two or more different array elements, more preferably at least 100 array elements, and most preferably at least 1,000 array elements, on a 1 cm2 substrate surface. The hybridization signal from each of the array elements is individually distinguishable. In a preferred embodiment, the array elements have attached thereto polynucleotide probes derived from GATA-1 sequences discussed above.

A “polynucleotide” refers to a chain of nucleotides. Preferably, the chain has from about 75 to 10,000 nucleotides, more preferably from about 100 to 3,500 nucleotides. An “oligonucleotide” refers to a chain of nucleotides extending from 2-75 nucleotides, and preferably 9-79 nucleotides. The term “probe” refers to a polynucleotide sequence capable of hybridizing with a target sequence to form a polynucleotide probe/target complex. A “target polynucleotide” refers to a chain of nucleotides to which a polynucleotide probe can hybridize by base pairing. In some instances, the sequences will be complementary (no mismatches) when aligned. In other instances, there may be up to a 10% mismatch.

The microarray can be used for large scale genetic or gene expression analysis of a large number of target polynucleotides. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

When the composition of the invention is employed as hybridizable array elements in a microarray, the array elements are organized in an ordered fashion so that each element is present at a distinguishable, and preferably specified, location on the substrate. In the preferred embodiments, because the array elements are at specified locations on the substrate, the hybridization patterns and intensities (which together create a unique expression profile) can be interpreted in terms of expression levels of particular genes and can be correlated with a particular disease or condition or treatment.

The composition comprising a plurality of polynucleotide probes can also be used to purify a subpopulation of mRNAs, cDNAs, genomic fragments and the like, in a sample. Typically, samples will include target polynucleotides of interest and other nucleic acids which may enhance the hybridization background; therefore, it may be advantageous to remove these nucleic acids from the sample. One method for removing the additional nucleic acids is by hybridizing the sample containing target polynucleotides with immobilized polynucleotide probes under hybridizing conditions. Those nucleic acids that do not hybridize to the polynucleotide probes are removed and may be subjected to analysis or discarded. At a later point, the immobilized target polynucleotide probes can be released in the form of purified target polynucleotides.

1. Microarray Production

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double stranded, the probes may be either sense or antisense strands. Where the target polynucleotides are single stranded, the probes are complementary single strands.

In one embodiment, the probes are cDNAs. The size of the DNA sequence of interest may vary and is preferably from 100 to 10,000 nucleotides, more preferably from 150 to 3,500 nucleotides.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233, 1980). Alternatively, the probes can be generated, in whole or in part, enzymatically.

Nucleotide analog can be incorporated into the probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The polynucleotide probes can be immobilized on a substrate. Preferred substrates are any suitable rigid or semi-rigid support including membranes,; filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Complementary DNA (cDNA) can be arranged and then immobilized on a substrate. The probes can be immobilized by covalent means such as by chemical bonding procedures or UV. In one such method, a cDNA is bound to a glass surface which has been modified to contain epoxide or aldehyde groups. In another case, a cDNA probe is placed on a polylysine coated surface and then UV cross-linked (Shalon et al., PCT publication WO95/35505, herein incorporated by reference). In yet another method, a DNA is actively transported from a solution to a given position on a substrate by electrical means (Heller et al., U.S. Pat. No. 5,605,662). Alternatively, individual DNA clones can be gridded on a filter. Cells are lysed, proteins and cellular components degraded, and the DNA coupled to the filter by UV cross-linking.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

2. Sample Preparation for Microarray Analysis

In order to conduct sample analysis, a sample containing target polynucleotides is provided. The samples can be any sample containing target polynucleotides and obtained from any bodily fluid (blood, urine, saliva, phlegm, gastric juices, etc.), cultured cells, biopsies, or other tissue preparations. In particularly preferred embodiments, the sample is selected from the group consisting of blood, an amniocentesis sample, somatically in utero fetal blood, and bone marrow.

DNA or RNA can be isolated from the sample according to any of a number of methods well known to those of skill in the art. For example, methods of purification of nucleic acids are described in Tijssen Laboratory Techniques in Biochemistry and Molecular Biology: Hybridization With Nucleic Acid Probes, Part I. Theory and Nucleic Acid Preparation, Elsevier, New York N.Y. 1993. In one case, total RNA is isolated using the TRIZOL reagent (Life Technologies, Gaithersburg Md.), and mRNA is isolated using oligo d(T) column chromatography or glass beads. Alternatively, when target polynucleotides are derived from an mRNA, the target polynucleotides can be a cDNA reverse transcribed from an mRNA, an RNA transcribed from that cDNA, a DNA amplified from that cDNA, an RNA transcribed from the amplified DNA, and the like. When the target polynucleotide is derived from DNA, the target polynucleotide can be DNA amplified from DNA or RNA reverse transcribed from DNA. In yet another alternative, the targets are target polynucleotides prepared by more than one method.

When target polynucleotides are amplified, it is desirable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single stranded DNA template. The second DNA strand is polymerized using a DNA polymerase and a RNAse which assists in breaking up the DNA/RNA hybrid. After synthesis of the double stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine, U.S. Pat. No. 5,514,545).

Quantitation controls may be included within the sample to assure that amplification and labeling procedures do not change the true distribution of target polynucleotides in a sample. For this purpose, a sample is spiked with a known amount of a control target polynucleotide and the composition of probes includes reference probes which specifically hybridize with the control target polynucleotides. After hybridization and processing, the hybridization signals obtained can be calculated accurately by comparison to the signal obtained for control target polynucleotide added to the sample.

Prior to hybridization, it may be desirable to fragment the nucleic acid target polynucleotides. Fragmentation improves hybridization by minimizing secondary structure and cross-hybridization to other nucleic acid target polynucleotides in the sample or noncomplementary polynucleotide probes. Fragmentation can be performed by mechanical or chemical means.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as 3H, 14C, 32P, 33P or 35S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes, and the like. Preferably, fluorescent markers absorb light above about 300 nm, preferably above 400 nm, and usually emit light at wavelengths at least greater than 10 nm above the wavelength of the light absorbed. Preferred fluorescent markers include fluorescein, phycoerythrin, rhodamine, lissamine, and C3 and C5 available from Amersham Pharmacia Biotech (Piscataway N.J.).

Labeling can be carried out during an amplification reaction, such as polymerase chain reactions and in vitro transcription reactions, or by nick translation or 5′ or 3′-end-labeling reactions. When the label may be incorporated after or without an amplification step, the label is incorporated by using terminal transferase or by phosphorylating the 5′ end of the target polynucleotide using, e.g., a kinase and then incubating overnight with a labeled oligonucleotide in the presence of T4 RNA ligase.

Alternatively, the labeling moiety can be incorporated after hybridization once a probe/target complex has formed.

3. Hybridization and Detection in Microarrays

Hybridization causes a denatured probe and a denatured complementary target to form a stable nucleic acid duplex through base pairing. Hybridization methods are well known to those skilled in the art (See, e.g., Ausubel, Short Protocols in Molecular Biology, John Wiley & Sons, New York N.Y., units 2.8-2.11, 3.18-3.19 and 4.6-4.9, 1997). Conditions can be selected for hybridization where an exactly complementary target and probes can hybridize, i.e., each base pair must interact with its complementary base pair. Alternatively, conditions can be selected where a target and probes have mismatches but are still able to hybridize. Suitable conditions can be selected, for example, by varying the concentrations of salt in the prehybridization, hybridization and wash solutions, by varying the hybridization and wash temperatures, or by varying the polarity of the prehybridization, hybridization or wash solutions.

Hybridization can be performed at low stringency with buffers, such as 6×SSPE with 0.005% Triton X-100 at 37° C., which permits hybridization between target and probes that contain some mismatches to form target polynucleotide/probe complexes. Subsequent washes are performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl or Triton X-100, or a blocking agent, such as salmon sperm DNA.

After hybridization, the microarray is washed to remove nonhybridized nucleic acids, and complex formation between the hybridizable array elements and the target polynucleotides is detected. Methods for detecting complex formation are well known to those skilled in the art. In a preferred embodiment, the target polynucleotides are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensity. The scanned image is examined to determine the abundance/expression level of each hybridized target polynucleotide. Typically, microarray fluorescence intensities can be normalized to take into account variations in hybridization intensities when more than one microarray is used under similar test conditions. In a preferred embodiment, individual probe/target hybridization intensities are normalized using the intensities derived from internal normalization controls contained on each microarray.

4. Microarray Expression Profiles

This section describes an expression profile using the composition of the invention. The expression profile can be used to detect changes in the expression of genes implicated in disease.

The expression profile includes a plurality of detectable complexes. Each complex is formed by hybridization of one or more nucleic acids of the present invention to one or more complementary target polynucleotides. At least one of the nucleic acids of the invention, and preferably a plurality thereof, is hybridized to a complementary target polynucleotide forming at least one, and preferably a plurality, of complexes. A complex is detected by incorporating at least one labeling moiety in the complex as described above. The expression profiles provide “snapshots” that can show unique expression patterns that are characteristic of the presence or absence of a disease or condition.

After-performing hybridization experiments and interpreting detected signals from a microarray, particular probes can be identified and selected based on their expression patterns. Such probe sequences can be used to clone a full length sequence for the gene or to produce a polypeptide.

The composition comprising a plurality of probes can be used as hybridizable elements in a microarray. Such a microarray can be employed in several applications including diagnostics, prognostics and treatment regimens, drug discovery and development, toxicological and carcinogenicity studies, forensics, pharmacogenomics, and the like.

In one situation, the microarray is used to monitor the progression of disease. Researchers can assess and catalog the differences in gene expression between healthy and diseased tissues or cells. By analyzing changes in patterns of gene expression, disease can be diagnosed at earlier stages, before the patient is symptomatic. The invention can also be used to monitor the efficacy of treatment. For some treatments with known side effects, the microarray is employed to “fine tune” the treatment regimen. A dosage is established that causes a change in genetic expression patterns indicative of successful treatment. Expression patterns associated with undesirable side effects are avoided. This approach may be more sensitive and rapid than waiting for the patient to show inadequate improvement, or to manifest side effects, before altering the course of treatment.

Alternatively, animal models which mimic a disease, rather than patients, can be used to characterize expression profiles associated with a particular disease or condition. This gene expression data is useful in diagnosing and monitoring the course of disease in a patient, in determining gene targets for intervention, and in testing treatment regimens.

Also, researchers can use the microarray to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to determine the molecular mode of action of a drug.

### F. EXAMPLES

The following examples present preferred embodiments and techniques, but are not intended to be limiting. Those of skill in the art will, in light of the present disclosure, appreciate that many changes can be made in the specific materials and methods which are disclosed and still obtain a like or similar result without departing from the spirit and scope of the invention.

### Example 1

### Assessment of Mutations in GATA-1 in DS-AMKL

Recent studies have shown that mutations in the N-terminal zinc finger of the X-linked gene GATA-1 cause a variety of congenital dyserythropoietic anemias and thrombocytopenias (Nichols, K. E., Crispino, J. D., Poncz, M., White, J. G., Orkin, S. H., Maris, J. M. and Weiss, M. J. (2000) Nat Genet, 24(3), 266-70; Freson, K., Devriendt, K., Matthijs, G., Van Hoof, A., De Vos, R., Thys, C., Minner, K., Hoylaerts, M. F., Vermylen, J. and Van Geet, C. (2001) Blood, 98(1), 85-92; Mehaffey, M. G., Newton, A. L., Gandhi, M. J., Crossley, M. and Drachman, J. G. (2001) Blood, 98(9), 2681-8; Yu, C., Niakan, K. K., Matsushita, M., Stamatoyannopoulos, G., Orkin, S. H. and Raskind, W. H. (2002) Blood, 100(6), 2040-5). Given that missense mutations in GATA-1 lead to congenital blood disorders, the inventors hypothesized that acquired mutations in GATA-1 might be involved in the pathogenesis of other hematopoietic diseases. Since GATA-1 is required for the proper growth and maturation of both erythroid cells and megakaryocytes (Orkin, S. H. (2000) Nat Rev Genet, 1(1), 57-64), and megakaryocytes that lack GATA-1 proliferate excessively (Shivdasani, R. A., Fujiwara, Y., McDevitt, M. A. and Orkin, S. H. (1997) Embo J, 16(13), 3965-73; Vyas, P., Ault, K., Jackson, C. W., Orkin, S. H. and Shivdasani, R. A. (1999) Blood, 93(9), 2867-75), the initial experiments focused on patients with acute erythroleukemia (AML-M6), acute megakaryoblastic leukemia (AMKL), other AML subtypes, myelodysplastic syndrome (MDS), and AML evolving from MDS.

Using single strand conformation polymorphism (SSCP) method, the DNA from bone marrow cells isolated from patients with these disorders was screened for mutations in GATA-1. Five coding exons of GATA-1 were amplified by PCR and the migration of the resulting single strand DNAs was monitored by electrophoresis. FIG. 2a depicts an example of the SSCP analysis of the first coding exon of GATA-1 (exon 2). While the majority of PCR products migrated similarly, three distinct altered migration products are present in this panel of samples (FIG. 2a, arrows). Sequencing of PCR products generated from the excised aberrant SSCP products revealed a silent mutation in two samples (AML-M6-8 and AMKL-1) and a four base pair insertion within exon 2 in the third sample, which was taken from an AMKL patient with Down syndrome (DS-AMKL-1; see FIG. 2b). As a consequence of this insertion, the reading frame of GATA-1 is disrupted after codon 62 and a stop codon is introduced six residues downstream of tyrosine 62 (FIG. 2b, FIG. 2c).

The analysis was extended to encompass 75 patients with myeloid leukemias, including six patients with DS-AMKL, four with non-DS AMKL, and 10 with AML-M6. Strikingly, mutations within exon 2 of GATA-1 were detected in all five of the additional DS-AMKL DNA samples (Table 1). Each of these mutations altered the reading frame and introduced a premature stop codon within the N-terminal activation domain (FIG. 2c and Table 2). In contrast, mutations were not detected in GATA-1 in DNAs from 21 healthy individuals, 4 patients with non-DS AMKL, or 32 patients with other subtypes of AML. In addition, DNA from 22 patients with either therapy-related AML (t-AML), AML with antecedent MDS, or pure MDS also was analyzed (Table 1). None of these samples harbored mutations in GATA-1. Significantly, mutations were not present at detectable levels in GATA-1 in bone marrow samples taken from DS patients with either acute lymphoblastic leukemia (1 patient) or other subtypes of AML (2 patients; Table 1). Finally, clinical remission samples taken from patients DS-AMKL-1 and DS-AMKL-2 did not have a mutation in GATA-1, confirming that the mutations are somatically acquired.

TABLE 1
Six of six Down syndrome AMKL
patients have GATA-1 mutations

| | | | Predicted |
|---|---|---|---|
| Sample Type | Samples | Mutations^{a} | Polymorphisms^{b} |
| DS-AMKL | 6 | 6 | 0 |
| AMKL (Non-DS) | 4 | 0 | 1 |
| AML-M6 | 10 | 0 | 1 |
| Other de novo AML | 32 | 0 | 2 |
| t-AML or MDS/AML^{c} | 21 | 0 | 1 |
| Pure MDS | 1 | 0 | 0 |
| DS-ALL | 1 | 0 | 0 |
| Other AML-DS | 2 | 0 | 0 |
| Healthy controls | 21 | 0 | 0 |

^{a}SSCP was used to screen for alterations in the five coding exons of GATA-1 in samples from the above patients. Each of the mutations introduces a premature stop codon within the sequences encoding the N-terminal activation domain of GATA-1.
^{b}In contrast, the predicted polymorphisms do not alter the GATA-1 protein.
^{c}Patients with therapy-related AML (t-AML; n = 4) or with MDS that progressed to AML (n = 17) are grouped together. None of these patients had megakaryoblastic leukemia.

### Example 2

### RUNX1/AML1 is not Mutated in the DS-AMKL Blasts

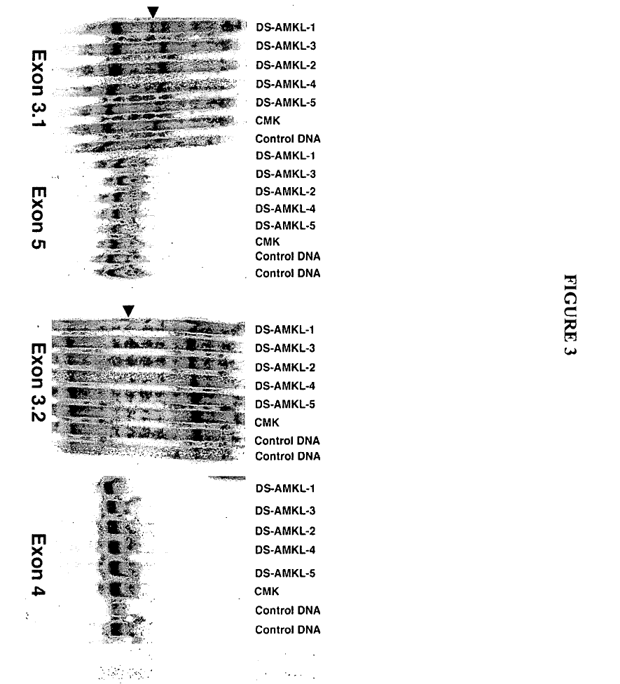

To ensure that the DS-AMKL leukemic process is specifically associated with the GATA-1 mutations, the inventors analyzed the sequences encoding the Runt domain of the essential transcription factor RUNX1 (AML1) in the DS-AMKL samples. Mutations in the Runt domain of RUNX1 have been implicated in a familial platelet disorder with predisposition to AML (Song, W. J., Sullivan, M. G., Legare, R. D., Hutchings, S., Tan, X., Kufrin, D., Ratajczak, J., Resende, I. C., Haworth, C., Hock, R., Loh, M., Felix, C., Roy, D. C., Busque, L., Kurnit, D., Willman, C., Gewirtz, A. M., Speck, N. A., Bushweller, J. H., Li, F. P., Gardiner, K., Poncz, M., Maris, J. M. and Gilliland, D. G. (1999) Nat Genet, 23(2), 166-75; Michaud, J., Wu, F., Osato, M., Cottles, G. M., Yanagida, M., Asou, N., Shigesada, K., Ito, Y., Benson, K. F., Raskind, W. H., Rossier, C., Antonarakis, S. E., Israels, S., McNicol, A., Weiss, H., Horwitz, M. and Scott, H. S. (2002) Blood, 99(4), 1364-72), in sporadic cases of AML (Osato, M., Asou, N., Abdalla, E., Hoshino, K., Yamasaki, H., Okubo, T., Suzushima, H., Takatsuki, K., Kanno, T., Shigesada, K., Ito, Y. (1999) Blood, 93(6), 1817-1824; Preudhomme, C., Warot-Loze, D., Roumier, C., Grardel-Duflos, N., Garand, R., Lai, J. L., Dastugue, N., Macintyre, E., Denis, C., Bauters, F Kerckaert, J. P., Cosson, A. and Fenaux, P. (2000) Blood, 96(8), 2862-9), and in myeloid malignancies with acquired trisomy 21 (Preudhomme, C., Warot-Loze, D., Roumier, C., Grardel-Duflos, N., Garand, R., Lai, J. L., Dastugue, N., Macintyre, E., Denis, C., Bauters, F., Kerckaert, J. P., Cosson, A. and Fenaux, P. (2000) Blood, 96(8), 2862-9). Five of the DS-AMKL samples were analyzed by SSCP and the complete Runt domain from the DS-AMKL-1 and DS-AMKL-2 samples was sequenced (FIG. 3). Of the five patient samples, only a single change in RUNX1 was found: a silent alteration in the wobble position of the codon for Val101 in DNA from patient DS-AMKL-1 (FIG. 3). Thus, the leukemic blasts of each DS-AMKL patient examined harbored truncating mutations in GATA-1, but none had mutations in RUNX1. This observation provides strong evidence that the mutations in GATA-1 are specifically associated with the malignancy.

TABLE 2
All mutations result in premature termination of GATA-1 within the N-terminal AD

| Patient | Sex/Age | | | Final GATA-1 |
|---|---|---|---|---|
| DS-AMKL | (yrs) | Cytogenetics^{a} | Mutation^{b} | residue^{c} |
| 1 | F/2 | 48, XX, +8, +21c[19]/47, XX, +21c[1] | 187-188 ins TACT | Tyr 62 |
| 2 | M/2 | 8, XY, +8, +21c[1]/48, idem, t(5; 6)(p15.3; p11.2) | 173-174 ins 15 bp | Tyr 62 |
| | | [5]/47, XY, +21c[14] | | |
| 3 | F/6 mo | 47, XX, add(7)(p11.2), +21c[12]/47, XX, +21c[8] | 187-188 ins TACT | Tyr 62 |
| 4 | M/3 | 49, XY, der(2)t(2; 5)(p22; q15), der(5)t(2; 5) | 152-210 del | Pro 50 |
| | | inv(5)(2pter −> 2p23::5p15.3 −> 5q15::2p22 −> | | |
| | | 2p23::5p15.3 −> 5pter), +8, +21, +21c[23]/47, | | |
| | | XY, +21c[7] | | |
| 5 | M/1 | 48, XY, +8, +21c[5]/49, idem, +21[7]/47, | 84-85 ins T | Thr 27 |
| | | XY, +21c[8] | | |
| 6 | M/3 | 47, XY, +21c[20] | 166-167 del | Ala 55 |

^{a}All patients had constitutional trisomy 21, and five of six had acquired chromosomal abnormalities in the leukemia cells.
^{b}4 insertions and 2 deletions were detected, all of which resulted in a shift of the reading frame of GATA-1. In each case, the terminal GATA-1 residue is located within the N-terminal activation domain.
^{c}Patients 1 and 3 are both female and have an identical insertion within GATA-1. This finding was confirmed both by multiple SSCP assays and by direct sequencing of exon 2 using DNA from bone marrow cells. Furthermore, analysis of microsatellite

### Example 3

### A DS-AMKL GATA-1 Mutant Allele Encodes for a Shortened, N-Terminal Deleted GATA-1 Protein

To determine whether any GATA-1 protein is translated by the mutated GATA-1 alleles, COS cells were transfected with a vector expressing either wild-type GATA-1, a mutant GATA-1 (Val205Gly) that fails to interact with its essential cofactor FOG-1 (Crispino, J. D., Lodish, M. B., MacKay, J. P. and Orkin, S. H. (1999) Mol Cell, 3(2), 219-28), or a DS representative GATA-1 mutant, Tyr63 Stop. Next, a variety of anti-GATA-1 antibodies were used to probe Western blots of nuclear extracts from the transfected cells. The N6 monoclonal antibody, which recognizes N-terminal residues surrounding codon 70, detected the 50 kD wild-type GATA-1 protein and the Val205Gly mutant protein, but no protein in the extracts from cells transfected with the Tyr63Stop mutant construct. (FIG. 4a, lanes 1-3). In contrast, the C-20 polyclonal antibody, raised against the C-terminus of GATA-1, detected a 40 kD protein in extracts from cells transfected with each of the DNAs (FIG. 4a, lanes 4-6). This 40 kD protein likely corresponds to GATA-1s, an alternate translation product that has previously been shown to be expressed in murine fetal liver cells and in the human K562 cell line (derived from an erythroid blast crisis of chronic myelogenous leukemia) (Calligaris, R., Bottardi, S., Cogoi, S., Apezteguia, I. and Santoro, C. (1995) Proc Natl Acad Sci USA, 92(25), 11598-602). GATA-1s initiates translation at Met84 in exon 3, downstream of the mutated Tyr63 codon and, thus, lacks the N-terminal transactivation domain, while retaining both zinc fingers and the entire C-terminus (FIG. 4b).

### Example 4

### GATA-1s, but not Full Length GATA-1, is Synthesized in the DS-AMKL Leukemic Blasts

To assay whether the 40 kD GATA-1 protein is produced in the leukemic cells of Down syndrome AMKL patients, a cell lysate was prepared from the one bone marrow sample where sufficient stored material was available. The cell lysate of patient DS-AMKL-1, a female, was then assayed for GATA-1 by Western blot analysis. Since leukemic cells in females show inactivation of the same X chromosome homolog due to monoclonality, and the mutant allele was only detected in leukemic cells, the inventors predicted that the wild-type allele would be on the inactive X chromosome and, hence, full length GATA-1 would not be translated. Full length GATA-1 (FIG. 4c, lanes 1 and 2) was not detected. However, the C-20 antibody recognized the 40 kD GATA-1 protein in the lysate from these leukemic cells (FIG. 4, lanes 1 and 2).

In addition, based on the findings that all six DS-AMKL patients examined harbored mutations in GATA-1, it was predicted that the human megakaryoblastic cell line CMK, which was established from the malignant cells of a male DS-AMKL patient (Komatsu, N., Suda, T., Moroi, M., Tokuyama, N., Sakata, Y., Okada, M., Nishida, T., Hirai, Y., Sato, T., Fuse, A. and et al. (1989) Blood, 74(1), 42-8; Sato, T., Fuse, A., Eguchi, M., Hayashi, Y., Ryo, R., Adachi, M., Kishimoto, Y., Teramura, M., Mizoguchi, H., Shima, Y. and et al. (1989) Br J Haematol, 72(2), 184-90), would also be deficient in GATA-1 production. Sequencing revealed the replacement of three nucleotides in exon 2 of GATA-1 by a single base, immediately following Met1, which places the sequence out of frame and introduces a stop codon within exon 2; As predicted, these cells fail to synthesize full length GATA-1, but do harbor trace amounts of the 40 kD protein (FIG. 4d) In contrast, the erythroid cell line HEL, the megakaryocytic cell line L8057, and, as previously demonstrated, K562 cells, all express the 50 kD version, but differ in the degree to which they generate the 40 kD protein. Taken together, these results demonstrate that DS-AMKL megakaryoblasts fail to express full length GATA-1, but continue to translate the 40 kD protein.

### Example 5

### GATA-1s has Reduced Transactivation Potential

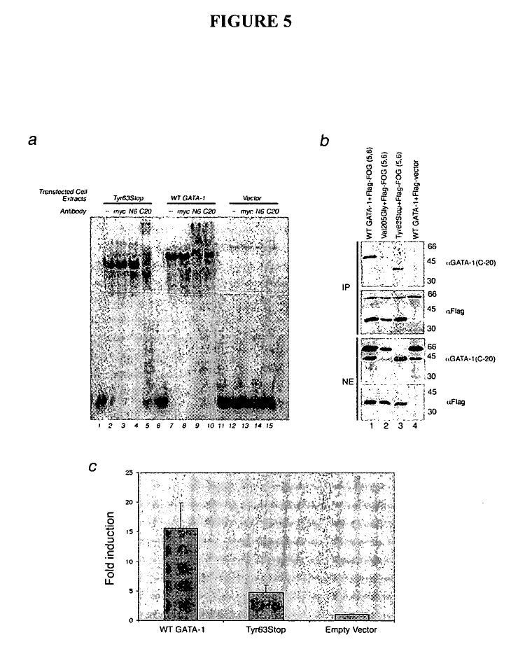

A series of functional assays were performed to study the activity of the 40 kD protein generated by the mutant GATA-1 allele. This alternate GATA-1 protein bound DNA efficiently, and was super-shifted by the C-20, but not the N6 antibody (FIG. 5a). It also dissociated from DNA at a rate similar to the full-length protein. Furthermore, the 40 kD GATA-1 interacted with the FOG-1 cofactor to the same extent as wild-type GATA-1 (FIG. 5b). However, as a result of the absence of the N-terminal activation domain (Martin, D. I. and Orkin, S. H. (1990) Genes Dev, 4(11), 1886-98), the 40 kD form has reduced transactivation potential (FIG. 5c), in accordance with previous observations (Shimizu, R., Takahashi, S., Ohneda, K., Engel, J. D. and Yamamoto, M. (2001) Embo J, 20(18), 5250-60; Calligaris, R., Bottardi, S., Cogoi, S., Apezteguia, I. and Santoro, C. (1995) Proc Nail Acad Sci US A, 92(25), 11598-602). Although a GATA-1 molecule that lacks its N-terminal activation domain can rescue differentiation of a GATA-1 deficient erythroid cell line (Weiss, M. J., Yu, C. and Orkin, S. H. (1997) Mol Cell Biol, 17(3), 1642-51), recent studies show that higher levels of ΔNt GATA-1 are required in vivo for rescue of GATA-1.05 knock-down mice (Shimizu, R., Takahashi, S., Ohneda, K., Engel, J. D. and Yamamoto, M. (2001) Embo J, 20(18), 5250-60). These rescued mice, which express the 40 kD protein at very high levels, do not exhibit any hematologic deficiencies, indicating that the shortened form of GATA-1 can substitute for full length GATA-1, but only when substantially overexpressed. The latter finding favors the model that, while the leukemic cells produce GATA-1 s, the levels are insufficient to promote proper development. Alternatively, there may be an essential function for the N-terminus in megakaryocytes.

It should be understood that the foregoing description relates to preferred embodiments of the invention and equivalents and variations that will be apparent to the reader are also intended as aspects of the invention. The references referred to herein throughout are incorporated by reference.

1. A method of diagnosing transient myeloproliferative disorder (TMD) comprising
(a) obtaining a sample from a subject suspect of having a predisposition to TMD; and
(b) determining the loss or mutation of a GATA-1 gene in cells of said sample,
wherein the loss or mutation of a GATA-1 gene, is diagnostic of TMD.

2-3. (canceled)

4. The method of claim 1, wherein said determining comprises assaying for a GATA-1 nucleic acid from said sample.

5. (canceled)

6. The method of claim 1, further comprising the step of comparing the expression of GATA-1 in said sample with the expression of GATA-1 in non-TMD samples.

7-8. (canceled)

9. The method of claim 1, wherein said determining comprises an assay selected from the group consisting of sequencing, wild-type oligonucleotide hybridization, mutant oligonucleotide hybridization, SSCP analysis, PCR, denaturing gradient gel electrophoresis and RNase protection.

10. The method of claim 9, wherein said evaluating comprises performing nucleic acid hybridization using an oligonucleotide derived from wild-type or mutant GATA-1 and said oligonucleotide is configured in an array on a chip or wafer.

11. The method of claim 1, wherein said TMD sample comprises a mutation in the coding sequence of GATA-1.

12. (canceled)

13. The method of claim 11, wherein said mutation is a frameshift mutation.

14. (canceled)

15. The method of claim 11, wherein said mutation is in exon 2.

16. The method of claim 13, wherein said frameshift results from a deletion in codons 1 through to 83.

17. The method of claim 16, wherein said frameshift results in a STOP at codon 62 of wild-type GATA-1.

18. The method of claim 12, wherein said mutation produces mutant GATA-1 protein that is a shortened GATA-1 protein which lacks all or a portion of the N-terminal activation domain of wild-type GATA-1 protein.

19. The method of claim 18, wherein said mutant GATA-1 protein interacts with friend of GATA-1 to the same extent as full-length GATA-1, but has a reduced transactivation potential.

20. (canceled)

21. The method of claim 15, wherein said mutation is an insertion mutation which disrupts the open reading frame of GATA-1 after codon 62.

22. The method of claim 21, wherein said disruption after codon 62 results in the introduction of a stop codon 6 residues after tyrosine 62.

23. The method of claim 21, wherein said insertion mutation is an insertion of TACT at 187-188 of GATA-1.

24. The method of claim 21, wherein said insertion mutation is a 15 base pair insertion at 173-174 of GATA-1.

25. The method of claim 20, wherein said insertion mutation is an insertion of T at 84-85 of GATA-1.

26. (canceled)

27. The method of claim 11, wherein said mutation is a deletion mutation in the open reading frame of GATA-1.

28. The method of claim 27, wherein said deletion mutation is a deletion of 152-210 of the open reading frame of GATA-1.

29. (canceled)

30. The method of claim 27, wherein said deletion mutation is a deletion of 166-167 of the open reading frame of GATA-1.

31. (canceled)

32. The method of claim 1, wherein the subject has been diagnosed with a Down syndrome.

33-54. (canceled)
